(19) United States
(12) Patent Application Publication
(10) Pub. No.: US 2025/0122273 A1
(43) Pub. Date: Apr. 17, 2025

(54) USE OF ANTI-SCLEROSTIN ANTIBODIES IN THE TREATMENT OF OSTEOGENESIS IMPERFECTA

(71) Applicant: MEREO BIOPHARMA 3 LIMITED, London (GB)

(72) Inventors: Uwe JUNKER, Basel (CH); Michaela KNEISSEL, Basel (CH); Anthony Kent HALL, Oegstgeest (NL); Rena Joy EUDY; Matthew Manning RIGGS

(73) Assignee: MEREO BIOPHARMA 3 LIMITED, London (GB)

(21) Appl. No.: 18/935,369

(22) Filed: Nov. 1, 2024

Related U.S. Application Data
(63) Continuation of application No. 17/187,168, filed on Feb. 26, 2021, now Pat. No. 12,173,058.
Application No. 17/187,168 is a continuation of application No. 16/469,920, filed on Jun. 14, 2019, now Pat. No. 10,961,305.
(60) Provisional application No. 62/437,358, filed on Dec. 21, 2016.

(51) Int. Cl. C07K 16/22; A61K 9/00; A61K 9/19; A61K 31/663; A61K 38/22; A61K 38/29; A61K 39/00; A61K 39/395; A61P 19/08

(52) U.S. Cl. CPC C07K 16/22; A61K 9/0019; A61K 9/19; A61K 31/663; A61K 39/3955; A61P 19/08; A61K 38/22; A61K 38/29; A61K 2039/505; A61K 2039/54; A61K 2039/545; C07K 2317/56; C07K 2317/565

(57) ABSTRACT

Disclosed are methods for treating a patient suffering from osteogenesis imperfecta comprising administering to the patient a therapeutically effective amount of an anti-sclerostin antibody. Methods for increasing bone formation and reducing bone resorption in an osteogenesis imperfecta patient by administering to the patient a therapeutically effective amount of an anti-sclerostin antibody are also disclosed. Further disclosed are compositions for increasing bone formation and reducing bone resorption in an osteogenesis imperfecta patient. The compositions comprise a therapeutically effective amount of an anti-sclerostin antibody. The invention also provides an anti-sclerostin antibody for use in the treatment of osteogenesis imperfecta.

### PRIORITY CLAIM

This application is a continuation of U.S. patent application Ser. No. 17/187,168, filed Feb. 26, 2021, which is a continuation of U.S. patent application Ser. No. 16/469,920, filed Jun. 14, 2019, which is a U.S. 371 application of International Application No. PCT/GB17/053849, filed Dec. 21, 2017, which claims priority to U.S. Provisional Application No. 62/437,358, filed Dec. 21, 2016, all of which are hereby incorporated by reference in their entireties.

### FIELD OF INVENTION

This invention relates to antibodies and their use as pharmaceutical compositions, more specifically to the use of anti-sclerostin antibodies in the treatment of osteogenesis imperfecta.

### SEQUENCE LISTING

The instant application contains a Sequence Listing which has been submitted in XML format via EFS-Web and is hereby incorporated by reference in its entirety. Said XML copy, created on Nov. 1, 2024, is named 122626-5001-US02.xml and is 202,328 kilobytes in size.

### BACKGROUND

Osteogenesis imperfecta (OI) is a rare genetic disorder of the connective tissue characterized by bone fragility and reduced bone mass. OI comprises a group of inherited disorders which primarily, but not always, arise from mutations in the genes encoding type I collagen. About 85% of the cases are linked to mutations in one of the two genes encoding type I collagen (COL1A1 and COL1A2). Clinically, OI is characterized by fragile bones that fracture easily and without any trauma. In addition, patients with OI are often affected by muscle weakness, hearing loss, fatigue, joint laxity, curved bones, scoliosis, blue sclerae, dentinogenesis imperfecta, and short stature.

The clinical classification system divides OI into types I-V. Type I OI patients usually suffer from a mild non-deforming disease that is often associated with a premature stop codon in COL1A1. This defect results in a reduced rate of type I collagen production and quantitatively less collagen in bone. Patients with type II OI usually die during the perinatal period, as a result of respiratory failure from multiple severe fractures that include the rib cage. Types III and IV OI are often associated with glycine substitution in COL1A1 and COL1A2,which is a qualitative defect that prevents the 3 polypeptide chains of type I collagen to intertwine properly to form a normal triple alpha helical structure. Type III OI is the most severe form of OI in those affected children who survive infancy, whereas patients with type IV have mild to moderate bone deformities. Type V is infrequent.

There remains a need for therapeutic methods and agents for the treatment of OI.

### BRIEF SUMMARY OF THE INVENTION

The inventors surprisingly found that human patients with osteogenesis imperfecta (OI) can successfully be treated with an anti-sclerostin antibody, as established by the examples, which confirm that the anti-sclerostin antibody BPS804 can both increase bone formation and reduce bone resorption in patients with OI. We disclosed high affinity, neutralizing, fully human anti-sclerostin monoclonal antibodies (collectively “the human anti-sclerostin monoclonal antibody”) and their potent in vitro activity and in vivo activity in our U.S. Pat. Nos. 7,879,322, 8,246,953, and 8,486,661, which are hereby incorporated in their entirety by reference thereto.

In one aspect, the present invention is a method for treating osteogenesis imperfecta (OI) in a human patient comprising administering to the human patient a therapeutically effective amount of an anti-sclerostin antibody comprising (a) a heavy chain variable region CDR1 comprising an amino acid sequence set forth in SEQ ID NO:4; (b) a heavy chain variable region CDR2 comprising an amino acid sequence set forth in SEQ ID NO:15; (c) a heavy chain variable region CDR3 comprising an amino acid sequence set forth in SEQ ID NO:26; (d) a light chain variable region CDR1 comprising an amino acid sequence set forth in SEQ ID NO:37; (e) a light chain variable region CDR2 comprising an amino acid sequence set forth in SEQ ID NO:48; and (f) a light chain variable region CDR3 comprising an amino acid sequence set forth in SEQ ID NO:59. This anti-sclerostin antibody is also referred to herein as ‘the antibody of the invention’ The anti-sclerostin antibody of the invention is administered intravenously at a dose of 1-50 mg per kg body weight of the human patient.

In one embodiment, the present invention is a method for increasing bone formation and reducing bone resorption in an osteogenesis imperfecta patient by administering to the patient a therapeutically effective amount of the anti-sclerostin antibody of the invention. In another aspect, the invention provides an anti-sclerostin antibody of the invention, for use in the treatment of osteogenesis imperfecta. In one embodiment the anti-sclerostin antibody increases bone formation and/or reduces bone resorption.

In another embodiment, the anti-sclerostin antibody comprises an immunoglobulin heavy chain variable domain (VH) having at least 90 (such as at least 95, 98, or 99 or 100) percent identity to the amino acid sequence set forth in SEQ ID NO: 70. In yet another embodiment, the anti-sclerostin antibody comprises an immunoglobulin light chain variable domain (VL) having at least 90 (such as at least 95, 98, or 99 or 100) percent identity to the amino acid sequence set forth in SEQ ID NO: 81. In still another embodiment, the anti-sclerostin antibody comprises a VH having at least 90 (such as at least 95, 98, or 99 or 100) percent identity to the amino acid sequence set forth in SEQ ID NO: 70, and a VL having at least 90 (such as at least 95, 98, or 99 or 100) percent identity to the amino acid sequence set forth in SEQ ID NO: 81. In yet another embodiment, the anti-sclerostin antibody comprises a VH having the amino acid sequence set forth in SEQ ID NO: 70, and a VL having the amino acid sequence set forth in SEQ ID NO: 81. In yet still another embodiment, the anti-sclerostin antibody comprises a heavy chain having at least 90 (such as at least 95, 98, or 99 or 100) percent identity to the amino acid sequence set forth in SEQ ID NO: 114 or 172, and/or at least 90 (such as at least 95, 98, or 99 or 100) percent identity to a light chain having the amino acid sequence set forth in SEQ ID NO: 125 or 173. In one embodiment, the anti-sclerostin antibody comprises a heavy chain having the amino acid sequence set forth in SEQ ID NO: 172 and a light chain having the amino acid sequence set forth in SEQ ID NO: 173.

In some embodiments, the therapeutically effective amount of the anti-sclerostin antibody of the invention is about 1-50 mg of said antibody per kg body weight of the human patient.

Another aspect of the invention is a pharmaceutical composition for increasing bone formation and reducing bone resorption in an osteogenesis imperfecta patient. In some embodiments, the composition contains an anti-sclerostin antibody comprising: a) at least one immunoglobulin heavy chain variable domain (VH) which comprises in sequence hypervariable regions a heavy chain variable region CDR1, a heavy chain variable region CDR2, and a heavy chain variable region CDR3, said heavy chain variable region CDR1 comprising an amino acid sequence set forth in SEQ ID NO: 4, said heavy chain variable region CDR2 comprising an amino acid sequence set forth in SEQ ID NO: 15, and said heavy chain variable region CDR3 comprising an amino acid sequence set forth in SEQ ID NO: 26; and b) at least one immunoglobulin light chain variable domain (VL) which comprises in sequence hypervariable regions a light chain variable region CDR1, a light chain variable region CDR2, and a light chain variable region CDR3, said light chain variable region CDR1 comprising an amino acid sequence set forth in SEQ ID NO: 37, said light chain variable region CDR2 comprising an amino acid sequence set forth in SEQ ID NO: 48, and said light chain variable region CDR3 comprising an amino acid sequence set forth in SEQ ID NO: 59.

In another embodiment, the anti-sclerostin antibody in the pharmaceutical composition comprises a VH having at least 95 percent identity to the amino acid sequence set forth in SEQ ID NO: 70. In yet another embodiment, the anti-sclerostin antibody comprises a VL having at least 95 percent identity to the amino acid sequence set forth in SEQ ID NO: 81.

In still another embodiment, the anti-sclerostin antibody comprises a VH having at least 95 percent identity to the amino acid sequence set forth in SEQ ID NO: 70, and a VL having at least 95 percent identity to the amino acid sequence set forth in SEQ ID NO: 81. In yet another embodiment, the anti-sclerostin antibody comprises a VH having the amino acid sequence set forth in SEQ ID NO: 70, and a VL having the amino acid sequence set forth in SEQ ID NO: 81. In yet still another embodiment, the anti-sclerostin antibody comprises a heavy chain having the amino acid sequence set forth in SEQ ID NO: 114 or 172, and/or a light chain having the amino acid sequence set forth in SEQ ID NO: 125 or 173.

In one aspect the invention provides a pharmaceutical composition comprising an anti-sclerostin antibody as disclosed herein. In one embodiment the pharmaceutical composition can be used in the methods/uses described herein

### BRIEF DESCRIPTION OF THE DRAWINGS

FIG. 1 shows a table with results of the one-sample t-test analysis results for the bone metabolism biomarkers.

FIG. 2 shows a table with results of the two-sample t-test analysis of the bone metabolism biomarkers.

FIG. 3 shows a graph depicting ratios to baseline in geometric means (plus or minus SD) for PINP.

FIG. 4 shows a graph depicting ratios to baseline in geometric means (plus or minus SD) for PICP.

FIG. 5 shows a graph depicting ratios to baseline in geometric means (plus or minus SD) for BSAP.

FIG. 6 shows a graph depicting ratios to baseline in geometric means (plus or minus SD) for OC.

FIG. 7 shows a graph depicting Bayesian posterior probabilities of ratios to baseline for BMD of lumbar spine (measured by DXA) data.

FIG. 8 shows a graph depicting ratios of baseline in geometric means (plus or minus SD) for CTX-1.

### DETAILED DESCRIPTION OF THE INVENTION

The present invention is based on the unexpected and surprising findings that human patients with osteogenesis imperfecta (OI) can successfully be treated with an anti-sclerostin antibody. Administration of an anti-sclerostin antibody to human OI patients increases bone formation and reduces bone resorption, as shown in the examples.

By way of background, sclerostin is a naturally occurring protein that in humans is encoded by the SOST gene. Sclerostin is a secreted glycoprotein with a C terminal cysteine knot-like (CTCK) domain and sequence similarity to the DAN (differential screening-selected gene aberrative in neuroblastoma) family of bone morphogenetic protein (BMP) antagonists.

As shown for the first time by the present inventors, inhibiting sclerostin with anti-sclerostin antibodies boosts bone formation and density and provides beneficial effects in treating OI in humans. Unlike previous anabolic treatments using GH or PTH which increase bone turnover, a treatment with an anti-sclerostin antibody offers the advantage of stimulating bone formation while inhibiting bone resorption in OI patients. The clinical utility of anti-sclerostin antibodies therefore includes the treatment of osteogenesis imperfecta in humans where bone formation is of therapeutic benefit in a disease characterized by a significant lack of bone matrix.

As such, the invention is directed to methods of using anti-sclerostin antibodies in the treatment of OI, as well as compositions comprising anti-sclerostin antibodies for use in treating OI. In one aspect the invention is concerned with the treatment of osteogenesis imperfecta types I, III or IV. Accordingly, the invention provides anti-sclerostin antibodies and pharmaceutical compositions comprising said antibodies for use in the treatment of OI, preferably OI types I, III or IV. The anti-sclerostin antibody of the invention is a human monoclonal anti-sclerostin antibody.

The methods and uses of the anti-sclerostin antibody in the present invention were unexpected and surprising because the antibody treatment in OI patients results in both an increase of bone formation and a reduction of bone resorption.

In order that the present invention may be more readily understood, certain terms are first defined. Additional definitions are set forth throughout the detailed description.

The term “comprising” encompasses “including” as well as “consisting” e.g. a composition “comprising” X may consist exclusively of X or may include something additional e.g. X+Y.

The term “about” in relation to a numerical value x means, for example, x±10%.

The term sclerostin refers to human sclerostin as defined in SEQ ID NO: 155. Recombinant human sclerostin can be obtained from R&D Systems (Minneapolis, Minn., USA; 2006 cat #1406-ST-025). Additionally, recombinant mouse sclerostin/SOST is commercially available from R&D Systems (Minneapolis, Minn., USA; 2006 cat #1589-ST-025). U.S. Pat. Nos. 6,395,511 and 6,803,453, and U.S. Patent Publications 20040009535 and 20050106683 refer to anti-sclerostin antibodies in general.

The term “antibody” as referred to herein includes whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In one embodiment, reference to an antibody herein embraces isolated, monoclonal, human and humanized monoclonal antibodies.

The term “antigen-binding portion” of an antibody (or simply “antigen portion”), as used herein, refers to full length or one or more fragments of an antibody that retain the ability to specifically bind to an antigen (e.g., sclerostin). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding region” of an antibody. These antibody fragments are obtained using conventional techniques known to those of skill in the art, and the fragments are screened for utility in the same manner as are intact antibodies.

An “isolated antibody”, as used herein, refers to an antibody that is substantially free of other antibodies having different antigenic specificities (e.g., an isolated antibody that specifically binds sclerostin is substantially free of antibodies that specifically bind antigens other than sclerostin). An isolated antibody that specifically binds sclerostin may, however, have cross-reactivity to other antigens, such as sclerostin molecules from other species. Moreover, an isolated antibody may be substantially free of other cellular material and/or chemicals. In one embodiment, reference to an antibody herein means an isolated antibody.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis as described in Knappik, et al. (2000. J Mol Biol 296, 57-86).

The human antibodies may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In one embodiment, and as used herein, an antibody that binds sclerostin (e.g. an anti-sclerostin antibody) means that it specifically binds to sclerostin polypeptide. “Specifically binds to sclerostin polypeptide” is intended to refer to an antibody that binds to sclerostin polypeptide with a K_{D} of 1×10⁻⁸ M or less, 1×10⁻⁹ M or less, or 1×10⁻¹⁰ M or less. The term “K_{D}”, as used herein, is intended to refer to the dissociation constant, which is obtained from the ratio of K_{d} to Kₐ (i.e. Kₐ/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system.

Standard assays to evaluate the binding ability of the antibodies toward sclerostin of various species are known in the art, including for example, ELISAs, western blots and RIAs. Suitable assays are described in detail in WO2009/047356. The binding kinetics (e.g., binding affinity) of the antibodies also can be assessed by standard assays known in the art, such as by Biacore analysis. Assays to evaluate the effects of the antibodies on functional properties of sclerostin (e.g., receptor binding, preventing or ameliorating osteolysis) are described in further detail in WO2009/047356.

As used herein, the percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al., 1990 J.Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., 1997 Nucleic Acids Res. 25(17):3389-3402. When utilizing BLAST and Gapped BLAST programs, the default parameters of the respective programs (e.g., XBLAST and NBLAST) can be used. See http:www.ncbi.nhn.nih.gov.

Various aspects of the invention are described in further detail in the following subsections.

### Methods of Treatment With and Uses of Anti-Sclerostin Antibodies in OI Treatment

In one aspect, the invention is directed to methods of treating OI patients with anti-sclerostin antibodies as described herein or uses of these anti-sclerostin antibodies in OI treatment. The treatment with anti-sclerostin antibodies may both increase bone formation and reduce bone resorption in the OI patients. In one aspect, the invention provides an anti-sclerostin antibody for use in the treatment of OI. Suitable anti-sclerostin antibodies for use in the treatment of OI are disclosed herein.

In some embodiments, the invention is a method for increasing bone formation and reducing bone resorption in an OI patient and the method comprises administering to the patient a therapeutically effective amount of an anti-sclerostin antibody. In one embodiment, treatment of an OI patient with an anti-sclerostin antibody of the invention increases bone formation and/or reduces bone resorption in said OI patient.

The anti-sclerostin antibody of the invention comprises: a heavy chain variable region CDR1 of SEQ ID NO: 4; a heavy chain variable region CDR2 of SEQ ID NO: 15; a heavy chain variable region CDR3 of SEQ ID NO: 26; a light chain variable region CDR1 of SEQ ID NO: 37; a light chain variable region CDR2 of SEQ ID NO: 48; and a light chain variable region CDR3 of SEQ ID NO: 59. In one embodiment, the CDR regions are delineated using the Kabat system (Kabat, E. A., et al., 1991 Sequences of Proteins of Immunological Interest, Fifth Edition, U.S. Department of Health and Human Services, NIH Publication No. 91-3242).

In a certain embodiment, the anti-sclerostin antibody comprises: a VH polypeptide amino acid sequence having at least 95 percent identity to the amino acid sequence set forth as SEQ ID NO: 70.

In a certain embodiment, the anti-sclerostin antibody comprises: a VL polypeptide amino acid sequence having at least 95 percent identity to the amino acid sequence set forth as SEQ ID NO: 81.

In a certain embodiment, the anti-sclerostin antibody comprises: a VH polypeptide amino acid sequence having at least 95 percent identity to the amino acid sequence set forth as SEQ ID NO: 70 and a VL polypeptide amino acid sequence having at least 95 percent identity to the amino acid sequence set forth as SEQ ID NO: 81.

In a certain embodiment, the anti-sclerostin antibody comprises: the VH polypeptide amino acid sequence set forth as SEQ ID NO: 70 and the VL polypeptide amino acid sequence set forth as SEQ ID NO: 81.

In a certain embodiment, the anti-sclerostin antibody comprises: the heavy chain polypeptide amino acid sequence set forth as SEQ ID NO: 114 or 172 and the light chain polypeptide amino acid sequence set forth as SEQ ID NO: 125 or 173.

In a preferred embodiment, the anti-sclerostin antibody is the antibody BPS804, which is a human anti-sclerostin monoclonal antibody. BPS804 comprises the following CDRs: heavy chain variable region CDR1 of SEQ ID NO: 4; heavy chain variable region CDR2 of SEQ ID NO: 15; heavy chain variable region CDR3 of SEQ ID NO: 26; light chain variable region CDR1 of SEQ ID NO: 37; light chain variable region CDR2 of SEQ ID NO: 48; and light chain variable region CDR3 of SEQ ID NO: 59. The VH and VL sequences of BPS804 comprise: the VH polypeptide amino acid sequence set forth as SEQ ID NO: 70 and the VL polypeptide amino acid sequence set forth as SEQ ID NO: 81. The heavy and light chain sequences of BPS804 comprise: the heavy chain polypeptide amino acid sequence set forth as SEQ ID NO: 172 and the light chain polypeptide amino acid sequence set forth as SEQ ID NO: 173.

Additional characteristics of the anti-sclerostin antibodies of the present invention, such as BPS804 are described in WO2009/047356, which disclosure, discussion and data is hereby incorporated by reference thereto. By way of example only, the antibodies of the invention may exhibit at least one of the following functional properties: the antibody blocks the inhibitory effect of sclerostin in a cell based wnt signaling assay, the antibody blocks the inhibitory effect of sclerostin in a cell based mineralization assay, the antibody blocks the inhibitory effect of sclerostin in Smad1 phosphorylation assay, the antibody inhibits binding of sclerostin to the LRP-6, and the antibody increases bone formation and mass and density. As noted above, these properties are described in detail in WO2009/047356.

In relation to an antibody that “blocks the inhibitory effect of sclerostin in a cell based wnt signaling assay”, this is intended to refer to an antibody that restores wnt induced signaling in the presence of sclerostin in a cell-based super top flash (STF) assay with an IC50 less than 1 mM, 100 nM, 20 nM, 10 nM or less. WO2009/047356 describes said wnt STF assay.

In relation to an antibody that “blocks the inhibitory effect of sclerostin in a cell based mineralization assay”, this is intended to refer to an antibody that restores BMP2 induced mineralisation in the presence of sclerostin in a cell-based assay with an IC50 less than 1 mM, 500 nM, 100 nM, 10 nM, 1 nM or less.

In relation to an antibody that “blocks the inhibitory effect of sclerostin in Smad1 phosphorylation assay”, this is intended to refer to an antibody that restores BMP6 induced Smad1 phosphorylation in the presence of sclerostin in a cell based assay with an IC50 less than 1 mM, 500 nM, 100 nM, 10 nM, 1 nM or less.

In relation to an antibody that “inhibits binding of sclerostin to the LRP-6”, this is intended to refer to an antibody that inhibits sclerostin binding to LRP-6 with a IC50 of 1 rnM, 500 nM, 100 nM, 10 nM, 5 nM, 3 nM, 1 nM or less.

In relation to an antibody that “increases bone formation and mass and density”, this is intended to refer to an antibody that is capable of reaching bone formation, mass and density at the level of daily intermittent treatment with high anabolic dose of PTH, such as a daily intermittent treatment with 100 μg/kg of hPTH.

In one embodiment, the anti-sclerostin antibody of the invention increases bone formation and/or reduces bone resorption.

### Dosage Regimen

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals. In one embodiment, the dosage unit form of the invention comprises 10-5000 mg of anti-sclerostin antibody, or 10-4000 mg, 10-3000 mg, 10-2000 mg, 10-1000 mg, 10-500 mg, 10-400 mg, 10-300 mg, 10-200 mg, 10-150 mg, 10-100 mg, 10-80 mg, 10-60 mg, 10-50 mg, 10-40 mg, 10-35 mg, 10-30 mg, 10-25 mg, 10-20 mg, or 10-15 mg. In one embodiment, the dosage unit form comprises 150 mg of anti-sclerostin antibody.

In one embodiment, the anti-sclerostin antibody of the dosage unit form is in a lyophilized state which may be in powder form. In another embodiment, the anti-sclerostin antibody of the dosage unit form is in solution. In one embodiment the dosage unit form of the invention is contained within a container such as a vial. In another embodiment, the container is a syringe.

In addition to the active substance (i.e. the anti-sclerostin antibody), the dosage unit form may comprise one or more additional substances and/or excipients. In one embodiment, the dosage unit form comprises one or more of the following: sucrose, arginine hydrochloride, L-histidine, polysorbate 80, hydrochloric acid and water for injection (wfi).

In one aspect, the invention provides a kit comprising an anti-sclerostin antibody of the invention or a pharmaceutical composition of the invention, or a lyophilizate of the invention, or a dosage unit form of the invention. Optionally the kit may further comprise instructions in the form of e.g. a patient information leaflet, instructions for reconstitution of the lyophilizate, and/or administration instructions. In one embodiment, the kit includes a syringe comprising one or more therapeutically effective doses of the anti-sclerostin antibody. The anti-sclerostin antibody in the syringe can be present in liquid or lyophilized form. The kit may further comprise a solution for reconstitution of the lyophilizate, and/or an infusion solution (e.g. dextrose 5% in sterile water).

For administration of the anti-sclerostin antibody, the dosage ranges from about 1 milligram of said antibody per kilogram body weight of the patient (herein referred to as “mg/kg” throughout this application) to 50 mg/kg, more usually about 1 to 30 mg/kg, and still more usually about 1 to 20 mg/kg. For example dosages can be about 5 mg/kg body weight, about 10 mg/kg body weight, about 20 mg/kg body weight, or within the range of about 5-20 mg/kg.

In another aspect of the invention, the anti-sclerostin antibody is administered at a dose of 1-50 mg per kg body weight of a patient, such as at 2-50, 3-50, 5-50, 8-50 mg/kg.

In one embodiment, the anti-sclerostin antibody is administered at a dose of 2-50 mg per kg body weight of a patient, such as at 2-45, 2-40, 2-35, 2-30 mg/kg.

In one embodiment, the anti-sclerostin antibody is administered at a dose of 3-50 mg per kg body weight of a patient, such as at 3-45, 3-40, 3-35, 3-30 mg/kg.

In one embodiment, the anti-sclerostin antibody is administered at a dose of 5-50 mg per kg body weight of a patient, such as at 5-45, 5-40, 5-35, 5-30 mg/kg.

In one embodiment, the anti-sclerostin antibody is administered at a dose of 8-50 mg per kg body weight of a patient, such as at 8-45, 8-40, 8-35, 8-30 mg/kg.

In one embodiment, the anti-sclerostin antibody is administered at a dose of 10-50 mg per kg body weight of a patient, such as at 10-45, 10-40, 10-35, 10-30 mg/kg.

In one embodiment, the anti-sclerostin antibody is administered at a dose of 11-50 mg per kg body weight of a patient, such as at 11-45, 11-40, 11-35, 11-30 mg/kg.

In one embodiment, the anti-sclerostin antibody is administered at a dose of 12-50 mg per kg body weight of a patient, such as at 12-45, 12-40, 12-35, 12-30 mg/kg.

In one embodiment, the anti-sclerostin antibody is administered at a dose of 15-50 mg per kg body weight of a patient, such as at 15-45, 15-40, 15-35, 15-30 mg/kg.

In one embodiment, the anti-sclerostin antibody is administered at a dose of 18-50 mg per kg body weight of a patient, such as at 18-45, 18-40, 18-35, 18-30 mg/kg.

In one embodiment, the anti-sclerostin antibody is administered at a dose of 20-50 mg per kg body weight of a patient, such as at 20-45, 20-40, 20-35, 20-30 mg/kg.

In one embodiment, the anti-sclerostin antibody is administered at a dose of 5-20 mg per kg body weight of a patient, such as at 8-20, 10-20, 12-20, 15-20 mg/kg.

In one embodiment, the anti-sclerostin antibody is administered at a dose of 10 mg per kg body weight of a patient, or at 1, 2, 3, 4, 5, 6, 7, 8, 9 mg/kg.

In one embodiment, the anti-sclerostin antibody is administered at a dose of 20 mg per kg body weight of a patient, or at 11, 12, 13, 14, 15, 17, 18, 19 mg/kg.

In one embodiment, the anti-sclerostin antibody is administered at a dose of 30 mg per kg body weight of a patient, or at 21, 22, 23, 24, 25, 26, 27, 28, 29 mg/kg.

In one embodiment, the anti-sclerostin antibody is administered at a dose of 10 mg per kg body weight of a patient. In a related embodiment, the anti-sclerostin antibody is administered at a dose of 8-12 mg/kg, or at 8-15 mg/kg.

In one embodiment, the anti-sclerostin antibody is administered at a dose of or 20 mg per kg body weight of a patient. In a related embodiment, the anti-sclerostin antibody is administered at a dose of 18-22 mg/kg, or at 15-25 mg/kg.

In another aspect of the invention, the anti-sclerostin antibody is administered at a dose of 10-5000 mg. In one embodiment, the anti-sclerostin antibody is administered at a dose of 10-4000 mg, 10-3000 mg, 10-2000 mg, 10-1000 mg, 10-500 mg, 10-400 mg, 10-300 mg, 10-200 mg, 10-150 mg, 10-100 mg, 10-80 mg, 10-60 mg, 10-50 mg, 10-40 mg, 10-35 mg, 10-30 mg, 10-25 mg, 10-20 mg, or 10-15 mg. In one embodiment, the anti-sclerostin antibody is administered at a dose of 10-3500 mg. In one embodiment, the anti-sclerostin antibody is administered at a dose of 10-3000 mg. In one embodiment, the anti-sclerostin antibody is administered at a dose of 10-2000 mg. In one embodiment, the anti-sclerostin antibody is administered at a dose of 10-1500 mg. In one embodiment, the anti-sclerostin antibody is administered at a dose of 10-1000 mg.

An exemplary treatment regime entails administration of multiple doses, which may be of the same dosage or different dosages ranging from, e.g., about 5-20 mg/kg, under a dosing schedule of once per week, once every two weeks, once every three weeks, once every four weeks, once a month or monthly, once every five weeks, once every six weeks, once every seven weeks, once every eight weeks, once every two months (i.e. bi-monthly), once every three months (i.e. quarterly), once every three to six months, semi-annually, or annually. In some embodiments, the multiple doses can be 2-20 doses, more usually 2-10 doses, and still more usually 3-5 doses, and still further more usually 3 doses under a dosing schedule of once per week, once every two weeks, once every three weeks, once every four weeks, once a month or monthly, once every five weeks, once every six weeks, once every seven weeks, once every eight weeks, once every two months (i.e. bi-monthly), once every three months, once every three to six months, semi-annually, or annually.

In another aspect of the invention, the anti-sclerostin antibody may be administered to a patient on a daily, weekly, bi-weekly, monthly, bi-monthly, quarterly or annual basis.

In one embodiment, the anti-sclerostin antibody is administered to a patient on a weekly basis. In another embodiment, administration occurs every 2, 3, 4, 5, 6, or 7 weeks.

In one embodiment, the anti-sclerostin antibody is administered to a patient on a monthly basis. In another embodiment, administration occurs every 2, 3, 4, 5 or 6 months.

In one embodiment, the anti-sclerostin antibody is administered to a patient every three months (i.e. on a quarterly basis). In the event of more frequent administration regimens, such as weekly or daily administration, an administration route that allows patients to self-administer is preferred. By way of example, a subcutaneous, topical or oral administration route may facilitate self-administration of the anti-sclerostin antibody and preclude visits to a doctor/hospital in order to receive treatment.

Another exemplary treatment regime entails administration of multiple doses, which may be of the same dosage or different dosages ranging from, e.g., about 5-20 mg/kg, until a treatment target is achieved or reached in the patient. The treatment target is achieved or reached after a certain number of doses are administered. The treatment target may be a complete normalization of bone mineral density, a partial normalization of bone mineral density, or a reduced frequency of bone fracture incidence, an increased level of bone formation markers, or a decreased level of bone resorption markers. Thus, in one embodiment, the invention provides an anti-sclerostin antibody for use in the treatment of OI, wherein the anti-sclerostin antibody reduces the fracture rate in a patient/patient population compared to a control patient/patient population. Preferably, the anti-sclerostin antibody reduces the fracture rate by at least 10, 20, 30, 35, 40, 50, 60, 70, 80, or 90 percent. In one embodiment the anti-sclerostin antibody reduces the fracture rate by at least 30 percent. In one embodiment, fractures are defined as peripheral or vertebral fractures (including all major, minor, and vertebral clinical fractures; fractures only detected by means of investigations without clinical symptoms are not included), confirmed by radiologic investigation(s). In one embodiment the fracture rate pertains to a population of patients. The patient population and control patient populations are preferably of a size that allow a statistically significant comparison to be made.

In one aspect of the invention, a treatment regimen entails a first dosing regimen optionally followed by a second dosing regimen. A dosing regimen includes the dose administered and the frequency of administration. These can be selected from any of the aforementioned doses and administration frequencies and can be varied according to the clinical requirements of a patient. The doses and administration frequencies disclosed above are hereby explicitly embraced in this aspect of the invention.

By way of example, in one embodiment, the first dosing regimen is 1-50 mg per kg body weight of a patient, or 2-50 mg/kg, or 3-50 mg/kg, or 5-50 mg/kg, or 8-50 mg/kg, or 3-30 mg/kg, 5-30 mg/kg, or 8-30 mg/kg, or 10-30 mg/kg, or 12-30 mg/kg, or 15-30 mg/kg, or 12-25 mg/kg, or 15-25 mg/kg administered on a monthly basis. In another exemplary embodiment, the first dosing regimen is 10-5000 mg administered on a monthly basis.

In one embodiment, the first dosing regimen is 1-50 mg per kg body weight of a patient administered on a monthly basis.

In one embodiment, the first dosing regimen is 2-50 mg per kg body weight of a patient administered on a monthly basis.

In one embodiment, the first dosing regimen is 2-30 mg per kg body weight of a patient administered on a monthly basis.

In one embodiment, the first dosing regimen is 3-50 mg per kg body weight of a patient administered on a monthly basis.

In one embodiment, the first dosing regimen is 3-30 mg per kg body weight of a patient administered on a monthly basis.

In one embodiment, the first dosing regimen is 5-30 mg per kg body weight of a patient administered on a monthly basis.

In one embodiment, the first dosing regimen is 5-25 mg per kg body weight of a patient administered on a monthly basis.

In one embodiment, the first dosing regimen is 12-25 mg per kg body weight of a patient administered on a monthly basis.

In one embodiment, the first dosing regimen is 20 mg per kg body weight of a patient administered on a monthly basis.

In another exemplary embodiment, the first dosing regimen is 1-50 mg per kg body weight of a patient, or 2-50 mg/kg, or 3-50 mg/kg, or 5-50 mg/kg, or 8-50 mg/kg, or 8-30 mg/kg, or 10-30 mg/kg, or 12-30 mg/kg, or 15-30 mg/kg, or 12-25 mg/kg, or 15-25 mg/kg administered on a quarterly basis. In another exemplary embodiment, the first dosing regimen is 10-5000 mg administered on a quarterly basis.

In one embodiment, the first dosing regimen is 2-30 mg per kg body weight of a patient administered on a quarterly basis.

In one embodiment, the first dosing regimen is 3-30 mg per kg body weight of a patient administered on a quarterly basis.

In one embodiment, the first dosing regimen is 5-30 mg per kg body weight of a patient administered on a quarterly basis.

In one embodiment, the first dosing regimen is 5-25 mg per kg body weight of a patient administered on a quarterly basis.

In one embodiment, the first dosing regimen is 12-25 mg per kg body weight of a patient administered on a quarterly basis.

In one embodiment, the first dosing regimen is 20 mg per kg body weight of a patient administered on a quarterly basis.

In one embodiment and by way of example only, the second dosing regimen 1-50 mg per kg body weight of a patient, or 2-50 mg/kg, or 3-50 mg/kg, or 5-50 mg/kg, or 8-50 mg/kg, or 3-30 mg/kg, 5-30 mg/kg, or 8-30 mg/kg, or 10-30 mg/kg, or 12-30 mg/kg, or 12-25 mg/kg, or 15-30 mg/kg, or 15-25 mg/kg administered on a monthly basis. In another exemplary embodiment, the second dosing regimen is 10-5000 mg administered on a monthly basis

. In one embodiment, the second dosing regimen is 1-50 mg per kg body weight of a patient administered on a monthly basis.

In one embodiment, the second dosing regimen is 2-50 mg per kg body weight of a patient administered on a monthly basis.

In one embodiment, the second dosing regimen is 2-30 mg per kg body weight of a patient administered on a monthly basis.

In one embodiment, the second dosing regimen is 3-50 mg per kg body weight of a patient administered on a monthly basis.

In one embodiment, the second dosing regimen is 3-30 mg per kg body weight of a patient administered on a monthly basis.

In one embodiment, the second dosing regimen is 5-30 mg per kg body weight of a patient administered on a monthly basis.

In one embodiment, the second dosing regimen is 5-25 mg per kg body weight of a patient administered on a monthly basis.

In one embodiment, the second dosing regimen is 12-25 mg per kg body weight of a patient administered on a monthly basis.

In one embodiment, the second dosing regimen is 20 mg per kg body weight of a patient administered on a monthly basis.

In another embodiment, the second dosing regimen 1-50 mg per kg body weight of a patient, or 2-50 mg/kg, or 3-50 mg/kg, or 5-50 mg/kg, or 8-50 mg/kg, or 3-30 mg/kg, 5-30 mg/kg, or 8-30 mg/kg, or 10-30 mg/kg, or 12-30 mg/kg, or 12-25 mg/kg, or 15-30 mg/kg, or 15-25 mg/kg administered on a bi-monthly basis. In another exemplary embodiment, the second dosing regimen is 10-5000 mg administered on a bi-monthly basis.

In one embodiment, the second dosing regimen is 1-50 mg per kg body weight of a patient administered on a bi-monthly basis.

In one embodiment, the second dosing regimen is 2-50 mg per kg body weight of a patient administered on a bi-monthly basis.

In one embodiment, the second dosing regimen is 2-30 mg per kg body weight of a patient administered on a bi-monthly basis.

In one embodiment, the second dosing regimen is 3-50 mg per kg body weight of a patient administered on a bi-monthly basis.

In one embodiment, the second dosing regimen is 3-30 mg per kg body weight of a patient administered on a bi-monthly basis.

In one embodiment, the second dosing regimen is 5-30 mg per kg body weight of a patient administered on a bi-monthly basis.

In one embodiment, the second dosing regimen is 5-25 mg per kg body weight of a patient administered on a bi-monthly basis.

In one embodiment, the second dosing regimen is 12-25 mg per kg body weight of a patient administered on a bi-monthly basis.

In one embodiment, the second dosing regimen is 20 mg per kg body weight of a patient administered on a bi-monthly basis.

In another exemplary embodiment, the second dosing regimen is 1-50 mg per kg body weight of a patient, or 2-50 mg/kg 3-50 mg/kg, or 5-50 mg/kg, or 8-50 mg/kg, or 8-30 mg/kg, or 10-30 mg/kg, or 12-30 mg/kg, or 12-25 mg/kg, or 15-30 mg/kg, or 15-25 mg/kg administered on a quarterly basis. In another exemplary embodiment, the second dosing regimen is 10-5000 mg administered on a quarterly basis.

In one embodiment, the second dosing regimen is 2-30 mg per kg body weight of a patient administered on a quarterly basis.

In one embodiment, the second dosing regimen is 3-30 mg per kg body weight of a patient administered on a quarterly basis.

In one embodiment, the second dosing regimen is 5-30 mg per kg body weight of a patient administered on a quarterly basis.

In one embodiment, the second dosing regimen is 5-25 mg per kg body weight of a patient administered on a quarterly basis.

In one embodiment, the second dosing regimen is 12-25 mg per kg body weight of a patient administered on a quarterly basis.

In one embodiment, the second dosing regimen is 20 mg per kg body weight of a patient administered on a quarterly basis.

In a related embodiment, the first dosing regimen is 20 mg per kg body weight of a patient administered on a monthly basis and the second dosing regimen is 20 mg per kg body weight of a patient administered on a bi-monthly or quarterly basis.

The time period of administration of the first and second dosing regimens can be varied according to the clinical requirements of a patient. Thus, the first dosing regimen is administered for a first time period and the second dosing regimen is administered for a second time period.

Thus, and by way of example, the first and second time periods can be 1 month, 6 months, 12 months or any other time period.

In one embodiment, an anti-sclerostin antibody can be initially administered to a patient on a monthly basis for a period of 1 year, followed by administration on a bi-monthly or quarterly basis for a period of at least 1 year (such as 2 or more years).

Thus, in one embodiment, the first dosing regimen can be 20 mg per kg body weight of a human patient administered on a monthly basis for a period of 1 year, and the second dosing regimen can be 20 mg per kg body weight of a human patient administered on a bi-monthly or quarterly basis for a period of at least 1 year (such as 2 or more years).

Off-drug periods i.e. periods in which the anti-sclerostin antibody is not administered are also contemplated by the present invention, depending on the clinical requirements of the patient. Thus, in one embodiment the treatment with the anti-sclerostin antibody is discontinued for one or more (such as 2, 3, 4, 5, 6, 8, 10, 12 or more) months or even one or more years.

Measurements of the targets are known in the art. For example, bone mineral density may be measured by dual-energy x-ray absorptiometry (DXA), single-energy x-ray absorptiometry (SXA), quantitative computed tomography (CT), and ultrasound. DXA is an x-ray technique that has become the standard for measuring bone density in the art. Though it can be used for measurements of any skeletal site, clinical determinations are usually made of the lumbar spine and hip. Portable DXA machines have been developed that measure the heel (calcaneus), forearm (radius and ulna), or finger (phalanges), and DXA can also be used to measure body composition. Consequently, it has become standard practice to relate the results to “normal” values using T-scores, which compare individual results to those in a young population that is matched for race and gender. Alternatively, Z-scores compare individual results to those of an age-matched population that is also matched for race and gender. Thus, for example, a 60-year-old woman with a Z-score of −1 (1 SD below mean for age) could have a T-score of −2.5 (2.5 SD below mean for a young control group).

Yet another exemplary treatment regime entails administration of multiple doses, which may be of the same dosage or different dosages ranging from, e.g., about 5-20 mg/kg, for a long term use without a specific timeline to stop the administration. This may be the treatment regime to follow when a dose is needed to maintain an improved symptom in a continuous basis.

In other embodiments, dosage regimens for the anti-sclerostin antibody include three doses at the same dosage, e.g., at 5 mg/kg body weight, 10 mg/kg body weight, or 20 mg/kg body weight by intravenous administration sequentially with an interval of 1-3 weeks, preferably 2 weeks between two consecutive doses. In other embodiments, dosage regimens for the anti-sclerostin antibody include three doses at the three different dosages, e.g., first at 5 mg/kg body weight, then 10 mg/kg body weight, and finally 20 mg/kg body weight by intravenous administration sequentially with an interval of 1-3 weeks, preferably 2 weeks between two consecutive doses. In still other embodiments, dosage regimens for the anti-sclerostin antibody include three doses at the three different dosages, e.g., first at 20 mg/kg body weight, then 10 mg/kg body weight, and finally 5 mg/kg body weight by intravenous administration sequentially with an interval of 1-3 weeks, preferably 2 weeks between two consecutive doses.

In some embodiments, the anti-sclerostin antibody of the invention and one or more monoclonal antibodies with different binding specificities are administered simultaneously or sequentially, in which case the dosage of each antibody administered falls within the ranges indicated. In one embodiment the one or more additional monoclonal antibodies are also anti-sclerostin antibodies. Antibody is usually administered on multiple occasions. Intervals between single dosages can be, for example, weekly, monthly, every two months (i.e. bi-monthly) every three months (i.e. quarterly) or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml. In one embodiment, dosage of anti-sclerostin antibody of the invention is adjusted to achieve a plasma antibody concentration of about 1-1000 μg/ml or about 25-300 μg/ml.

Alternatively, in some embodiments, the anti-sclerostin antibody of the invention can be administered to OI patients as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

In some embodiments, actual dosage levels of the anti-sclerostin antibody may be varied so as to obtain an amount of the anti-sclerostin antibody which is effective to achieve the desired therapeutic response for a particular OI patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular anti-sclerostin antibody being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective amount” of the anti-sclerostin antibody may result in a decrease in severity of disease symptoms, an increase in frequency and duration of disease symptom-free periods, or a prevention of impairment or disability due to the disease affliction.

A composition of the anti-sclerostin antibody can be administered intravenously using one or more of a variety of methods known in the art. Administration for antibodies of the invention occurs via the intravenous route. In one embodiment, administration occurs intravenously by way of an infusion.

The anti-sclerostin antibody can be prepared with carriers that may protect the compound against rapid release, such as a controlled release formulation. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

### Patient Group

In one embodiment, the methods and uses described herein are for treating osteogenesis imperfecta using anti-sclerostin antibodies described herein. OI is classified by the genetics and severity of disease, and can be classified as type I OI, type II OI, type III OI, type IV OI, or type V OI according to the classification of Van Dijk and Sillence (2014, Am J Med Genet Part A 164A:1470-1481 and Van Dijk and Sillence, 2014, Am J Med Genet Part A 167A:1178; which are incorporated in their entirety by reference thereto). Classification relies on a combination of clinical evaluation/diagnosis, biochemical analysis as well as molecular genetic testing, and is routine for those skilled in the art. The OI nomenclature as used herein is as proposed by Van Dijk and Sillence, as referenced in the publications above.

In 80%-90% of people with OI, OI is caused by mutations in the COL1A1 and COL1A2 genes (17q21.33 and 7q22.3, respectively) encoding the alpha1 and alpha 2 chains of type-I collagen. A comprehensive database of over 1000 known mutations has been published along with a genotype-phenotype correlation (https://oi.gene.le.ac.uk/home.php; accessed 12 Dec. 2016). Mutations in other genes, such as CRTAP, LEPRE1 or PPIB, are also known. Molecular genetic tests for mutations in i.a. the COL1A1 and COL1A2 genes are known and routine for those skilled in the art. By way of example, Korkko et al. (1998) describe PCR amplification of the COL1A1 gene and the COL1A2 genes followed by mutation scanning by conformation-sensitive gel electrophoresis(CSGE) (Am. J. Hum. Genet. 62:98-110, 1998). van Dijk et al. (2010) describe COL1A1 mutation detection by a multiplex ligation-dependent probe amplification (MLPA) technique (Genet Med 12(11):736-741). More recently, Árvai, K. et al. (2016) describe next-generation sequencing methods (Sci. Rep. 6, 28417). These references are hereby incorporated by reference thereto.

In one embodiment, the methods and uses described herein are for treating patients who exhibit a deficiency of type-I collagen, e.g. OI types I-IV. As a result, the normal architecture of bone, consisting of collagen fibrils and hydroxyapatite crystals, is altered and causes brittleness. In one embodiment, the methods and uses herein are for treating human OI patients characterized by one or more mutations in COL1A1 and/or COL1A2.

In one embodiment, the methods and uses described herein are for treating OI type I, III and/or IV. In one embodiment, OI type I, III and IV are confirmed by DNA testing i.e. detection of COL1A1/COL1A2 mutations. Thus, in one embodiment the methods and uses herein are for treating OI type I, III and/or IV characterized by one or more mutations in COL1A1 and/or COL1A2.

In some embodiments, the methods and uses of the anti-sclerostin antibody of the invention are for treating a mild to moderate form of OI. In other embodiments of the methods and uses of the anti-sclerostin antibody, the patient under treatment has a type I OI, a type II OI, a type III OI, or a type IV OI. In still other embodiments of the methods and uses of the anti-sclerostin antibody, the OI patients are adult patients aged 18 and above. In yet still other embodiments of the methods and uses of the anti-sclerostin antibody, the OI patients are pediatric patients. Pediatric patients as defined herein embraces children aged 0-17 such as those aged 2-17, 3-17, 4-17 or 5-17. The term patients, as used herein, means human patients.

### Pharmaceutical Compositions

In another aspect, the present invention provides a pharmaceutical composition for increasing bone formation and reducing bone resorption in a patient suffering from osteogenesis imperfecta, which composition contains the anti-sclerostin antibody as described above. The pharmaceutical composition may be formulated with a pharmaceutically acceptable carrier. In one aspect, the invention provides a pharmaceutical composition comprising an anti-sclerostin antibody as disclosed herein. In one embodiment, the pharmaceutical composition comprising the anti-sclerostin antibody is for use in treating OI.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the anti-sclerostin antibody, may be coated in a material to protect the compound from the action of acids and other natural conditions that may inactivate the compound.

These compositions may also contain adjuvants such as preservatives, wetting agents, emulsifying agents and dispersing agents. Prevention of presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof. Thus, in one embodiment, the anti-sclerostin antibody of the invention/pharmaceutical composition of the invention is formulated as a lyophilizate powder. In a related embodiment, the lyophilizate is reconstituted prior to administration. Suitable liquids for reconstitution include water for injection (wfi).

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Pharmaceutical compositions of the invention and antibodies of the invention may also be administered in a combination therapy, i.e., combined with other active agents. For example, the combination therapy can include an anti-sclerostin antibody of the present invention combined with at least one other anti-inflammatory or anti-osteoporotic agent. Examples of therapeutic agents that can be used in combination therapy include bisphosphonates (such as alendronate, risedronate sodium, ibandronic acid, zoledronic acid, olpadronate, neridronate, skelid, bonefos), parathyroid hormone (e.g. teriparatide (rdna origin) injection), calcilytics, calcimimetics (e.g., cinacalcet), statins, anabolic steroids, lanthanum and strontium salts, and sodium fluoride. Thus, in one embodiment the anti-sclerostin antibodies of the invention can be administered in combination with one or more agents selected from: a calcitonin or an analogue or derivative thereof, e.g. salmon, eel or human calcitonin, calcilytics, calcimimetics (e.g., cinacalcet), a steroid hormone, e.g. an estrogen, a partial estrogen agonist or estrogen-gestagen combination, a SERM (Selective Estrogen Receptor Modulator) e.g. raloxifene, lasofoxifene, bazedoxifene, arzoxifene, FC1271, Tibolone (Livial®), a SARM (Selective Androgen Receptor Modulator), a RANKL antibody (such as denosumab), a cathepsin K inhibitor, vitamin D or an analogue thereof or PTH, a PTH fragment or a PTH derivative e.g. PTH (1-84) (such as Preos™ (parathyroid hormone 1-84)), PTH (1-34) (such as Forteo™ (teriparatide (rdna origin) injection)), PTH (1-36), PTH (1-38), PTH (1-31)NH2 or PTS 893. According to another embodiment, the antibodies of the invention may be employed in combination with other current osteoporosis therapy approaches, including bisphosphonates (e.g., Fosamax™ (alendronate), Actonel™ (risedronate sodium), Bonviva™ (ibandronic acid), Zometa™ (zoledronic acid), Aclasta™/Reclast™ (zoledronic acid), olpadronate, neridronate, skelid, bonefos), statins, anabolic steroids, lanthanum and strontium salts, and sodium fluoride.

In one embodiment, the antibodies of the invention may be administered in combination with an LRP4 modulating agent, i.e., an agent modulating the expression or activity of LRP4, e.g, an LRP4 neutralizing antibody.

In one embodiment, the antibodies of the invention may be administered in combination with an LRP5 modulating agent, i.e., an agent modulating the expression or activity of LRP5, e.g, an LRP5 neutralizing antibody.

In another embodiment, the antibodies of the invention may be administered in combination with a DKK1 modulating agent, i.e., an agent that interfere or neutralize Dkk-1 mediated antagonism of Wnt signaling, e.g., a DKK1 neutralizing antibody.

In one embodiment, the antibodies of the invention may be administered in combination with a bisphosphonate e.g. alendronate, risedronate sodium, ibandronic acid, zoledronic acid, zoledronic acid, olpadronate, neridronate, skelid, bonefos.

In one embodiment, the antibodies of the invention may be administered in combination with (i) zoledronic acid, (ii) an anti-DKK1 antibody, (iii) alendronate, (iv) an anti-LRP4 antibody, (v) hPTH and/or (vi) parathyroid hormone releasing agents (calcilytics).

Other agents which can be administered in combination with the anti-sclerostin antibodies of the invention include vitamin D and/or calcium. In one embodiment the vitamin D and/or calcium is administered if the patient has a vitamin D and/or calcium deficiency.

In one embodiment, the antibodies of the invention are administered together with another agent (e.g. the above mentioned agents), in a sequential manner (i.e. one after the other) or simultaneously. In one embodiment, the anti-sclerostin antibody is administered according to the aforementioned doses and frequencies of administration. Suitable doses for the combination therapy agent can be varied according to the clinical requirements of the patient.

The compositions are preferably formulated at physiological pH.

Therapeutic compositions can be administered with medical devices known in the art. For example, in one embodiment, a therapeutic composition of the invention can be administered with a needleless hypodermic injection device, such as the devices shown in U.S. Pat. Nos. 5,399,163; 5,383,851; 5,312,335; 5,064,413; 4,941,880; 4,790,824 or 4,596,556. Examples of well-known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicaments through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. No.4,439,196, which shows an osmotic drug delivery system having multi-chamber compartments; and U.S. Pat. No. 4,475,196, which shows an osmotic drug delivery system. These patents are incorporated herein by reference. Many other such implants, delivery systems, and modules are known to those skilled in the art. In one embodiment, the therapeutic composition of the invention can be administered with a syringe.

In certain embodiments, the antibodies of the invention can be formulated to ensure proper distribution in vivo.

In one aspect the invention provides use of an anti-sclerostin antibody for the manufacture of a medicament for the treatment of osteogenesis imperfecta. All of the other aspects/embodiments described herein apply equally to this particular aspect of the invention.

In another aspect the invention provides an anti-sclerostin antibody for use in the treatment of osteogenesis imperfecta. All of the other aspects/embodiments described herein apply equally to this particular aspect of the invention.

In one aspect, the invention provides an anti-sclerostin antibody for use in a clinical trial for osteogenesis imperfecta comprising comparing the number of fractures that have occurred in the clinical trial population at an interim time point during the clinical trial period with the number of fractures expected at said interim time point for said clinical trial population.

In a related aspect, the invention provides a method of conducting a clinical trial for osteogenesis imperfecta with an anti-sclerostin antibody comprising comparing the number of fractures that have occurred in a clinical trial population at an interim time point during the clinical trial period with the number of fractures expected at said interim time point for said clinical trial population. In one embodiment of the aforementioned aspects, the aspect may further comprise recruiting additional patients to the clinical trial population if the number of fractures in the clinical trial population at the interim time point is lower than the expected number of fractures for said clinical trial population. In one embodiment, the clinical trial period is extended if the number of fractures in the clinical trial population at the interim time point is lower than the expected number of fractures for said clinical trial population. In another embodiment, the clinical trial period is shortened if the number of fractures in the clinical trial population at the interim time point is higher than the expected number of fractures for said clinical trial population. The clinical trial population is made up of patients receiving the anti-sclerostin antibody and a control group of patients receiving a placebo. An advantage of these aspects/embodiments is that it allows the clinical trial period to be varied in response to developments in the clinic, which ultimately leads to a more efficient and cost-effective clinical trial which also benefits the patients. The expected number of fractures can be calculated from a baseline fracture rate which can be derived from historical data and/or from data from the clinical trial population prior to commencement of the clinical trial period. In one embodiment, the anti-sclerostin antibody is an anti-sclerostin antibody as defined herein. The interim time point can be varied and selected according to when a statistically significant comparison can be made. The clinical trial period begins with the administration of the anti-sclerostin antibody or placebo and ends once the final dose of anti-sclerostin antibody/placebo has been administered and the relevant data collected.

Sequence of BPS804 H-chain (SEQ ID NO: 172):
QVQLVESGGGLVQPGGSLRLSCAASGFTFRSHWLSWVRQAPGKGLEWVSN
INYDGSSTYYADSVKGRFTISRDNSKNTLYLQMNSLRAEDTAVYYCARDT
YLHFDYWGQGTLVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPVAGPSVFLFPPKPKDTLMIS
RTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK
Sequence of BPS804 L-chain (SEQ ID NO: 173):
DIALTQPASVSGSPGQSITISCTGTSSDVGDINDVSWYQQHPGKAPKLMI
YDVNNRPSGVSNRFSGSKSGNTASLTISGLQAEDEADYYCQSYAGSYLSE
VFGGGTKLTVLGQPKAAPSVTLFPPSSEELQANKATLVCLISDFYPGAVT
VAWKADSSPVKAGVETTTPSKQSNNKYAASSYLSLTPEQWKSHRSYSCQV
THEGSTVEKTVAPTECS

### MODES FOR CARRYING OUT THE INVENTION

### Example 1

This example describes a clinical trial to assess the use of an anti-sclerostin antibodyin the treatment of adult patients with OI. The patients were treated with three sequential intra-patient escalating doses of anti-sclerostin antibody BPS804, given as intravenous infusions separated by 2 weeks from each dose. An untreated reference group was enrolled as well for monitoring and observation of the natural OI disease progression with regard to changes in bone biomarker profiles. This trial was a randomized, open-label, intra-patient dose escalating study with an untreated reference group in 14 adult patients with moderate OI. Patients were randomized to the treatment group or the reference group at a ratio of 2:1.

Patients were administered every two weeks with escalating doses of the anti-sclerostin antibody: Week 1:5 mg/kg, Week 3:10 mg/kg and Week 5:20 mg/kg. The treatment period was followed by an about 3.6 month follow-up period. Patients who were randomized to the untreated reference group at screening were only admitted to the study site at week 7, study Day 43 and at the end of the study.

A description of the study drug BPS804 is presented in Table 1. BPS804 solution for infusion was administered as an infusion at a flow rate of about 2 mL/min until the desired dose has been delivered.

Biomarkers of bone metabolism including procollagen I N-terminal propeptide (PINP), procollagen I C-terminal propeptide (PICP), bone-specific alkaline phosphatase (BSAP), osteocalcin (OC), and bone mineral density (BMD) (measured by DXA) were assayed at baseline, day 43 and day 141.

TABLE 1
Study Drug BPS804

| Name | BPS804 |
|---|---|
| Formulation | Powder for solution for infusion |
| | (lyophilizate in vial) |
| Appearance before reconstitution | White lyophilizate cake |
| Appearance after reconstitution | Opalescent to clear, colorless |
| | solution |
| Unit dose | 150 mg per vial * |
| Packaging | 6 mL Type I glass vials |
| Diluent for iv administration | Dextrose 5% in water |
| | (USP or equivalent) in 250 mL |

* The vials contain a 20% overfill to allow a complete withdrawal of the labeled amount of BPS804.

Of the 14 patients enrolled in the study, nine patients were exposed to anti- sclerostin antibody treatment. The overall mean age of the patients was marginally higher in the anti-sclerostin antibody group (30.7 years) when compared to the reference group (27.4 years). The overall mean weight and height between the two groups were very similar. Summary of patient demographics is presented in Table 2. The overall median scores of the lumbar spine z-score were comparatively lower in the anti-sclerostin antibody group. The overall years on bisphosphonates cannot be compared between the groups considering the relative size of the number of the patients in each group. See Table 3.

TABLE 2
Demographic Summary By Treatment Group

| | BPS804 | Reference | Total |
|---|---|---|---|
| | N = 9 | N = 5 | N = 14 |
| Age (years) | | | |
| Mean (SD) | 30.7 (13.47) | 27.4 (15.47) | 29.5(13.71) |
| Median | 25.0 | 21.0 | 21.5 |
| Range | 19, 57 | 19, 55 | 19, 57 |
| Sex-n (%) | | | |
| Male | 7 (77.8) | 3 (60.0) | 10 (71.4) |
| Female | 2 (22.2) | 2 (40.0) | 4 (28.6) |
| Predominant | | | |
| Race-n (%) | | | |
| Caucasian | 9 (100) | 5 (100) | 14 (100) |
| Ethnicity-n (%) | | | |
| Mixed ethnicity | 1 (11.1) | 1 (20.0) | 2 (14.3) |
| Other | 8 (88.9) | 4 (80.0) | 12 (85.7) |
| Weight (kg)* | | | |
| Mean (SD) | 61.84 (14.378) | 58.20 (13.034) | 60.54 (13.519) |
| Median | 63.90 | 54.00 | 59.45 |
| Range | 43.5, 80.1 | 44.0, 75.0 | 43.5, 80.1 |
| Height (cm)* | | | |
| Mean (SD) | 161.6 (12.19) | 162.8 (13.85) | 162.0 (12.28) |
| Median | 162.0 | 161.0 | 162.0 |
| Range | 142, 178 | 142, 176 | 142, 178 |

*Weight and height are taken from Screening vital signs evaluations.

TABLE 3
Disease Characteristics By Treatment Group At Study Entry

| | BPS804 | Reference | Total |
|---|---|---|---|
| | N = 9 | N = 5 | N = 14 |
| Lumbar spine z-score* | | | |
| Mean (SD) | −2.59 (1.191) | −2.18 (0.514) | −2.44 (0.997) |
| Median | −2.30 | −2.07 | −2.19 |
| Range | −4.9, −1.1 | −2.9, −1.5 | −4.9, −1.1 |
| Yrs. on bisphosphonates# | | | |
| N | 2 | 1 | 3 |
| Mean (SD) | 8.53 (4.882) | 15.46 (—) | 10.84 (5.283) |
| Median | 8.53 | 15.46 | 11.99 |
| Range | 5.1, 12.0 | 15.5, 15.5 | 5.1, 15.5 |
| Subjects with D43 Biomarker data-n (%) | 9(100) | 5(100) | 14(100) |
| Subjects with D141 BMD data-n (%) | 9(100) | 4(80.0) | 13(92.9) |

*Lumbar spine z-score is taken at screening.
#Years on bisphosphonates is calculated from the medical history page, by taking the difference between the earliest start date of bisphosphonates medication and date of screening.

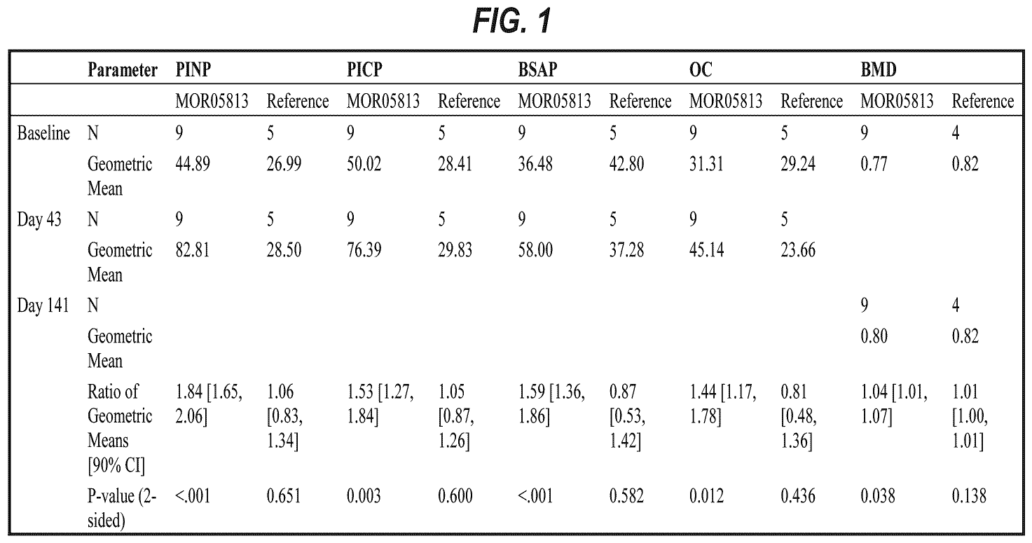

The assay results of the bone metabolism biomarkers for the study are presented in FIG. 1. The ratios of geometric means for PINP, PICP, BSAP, and OC at Day 43 were 1.84, 1.53, 1.59 and 1.44 with P-values of <0.001, 0.003, <0.001, and 0.012 in the BPS804 group. The ratio of geometric means for BMD at Day 141 was 1.04 with P-value of 0.038.

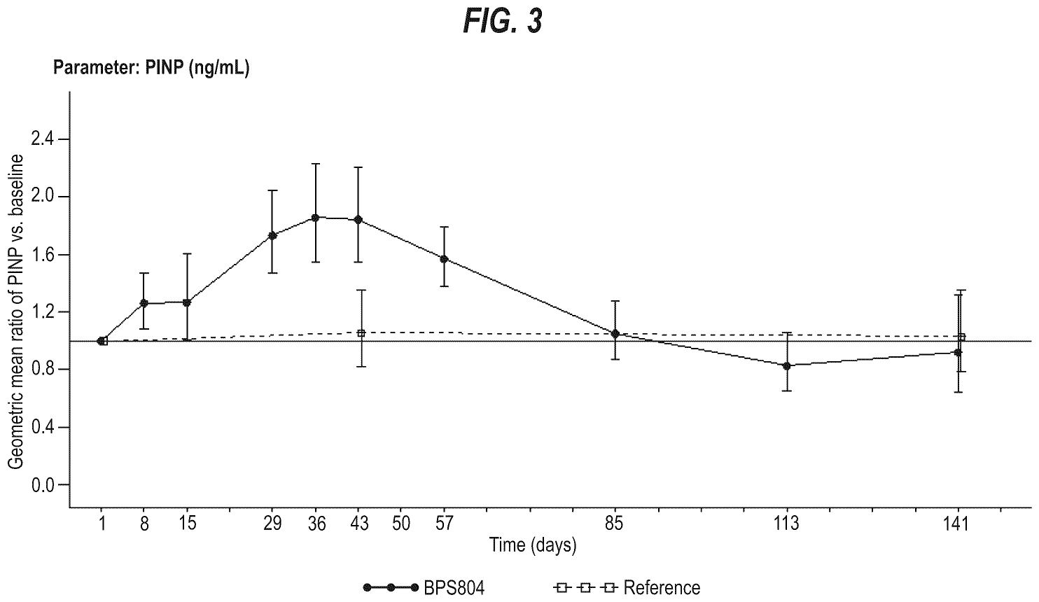

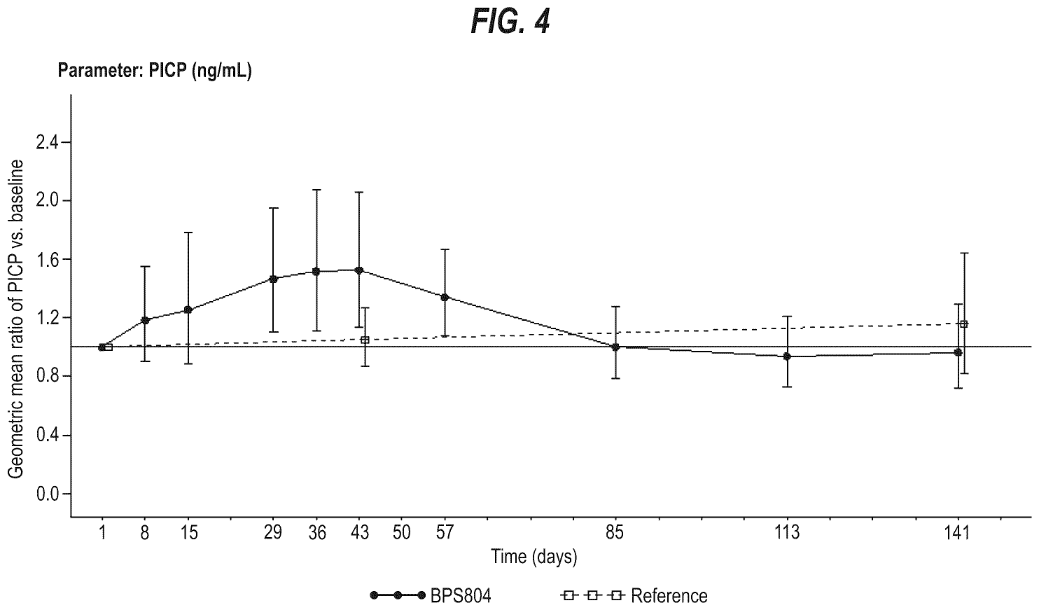

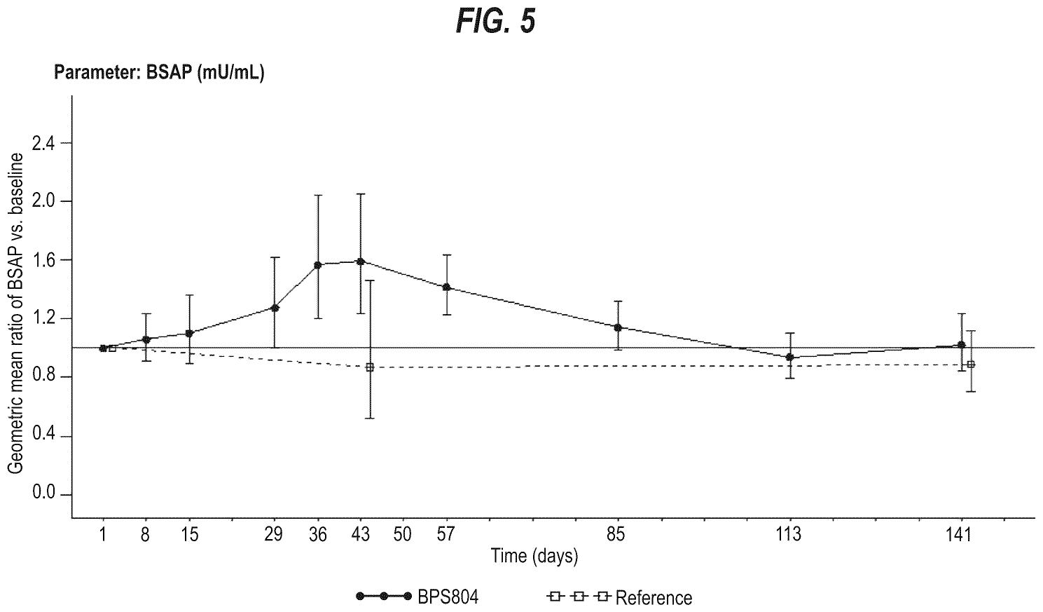

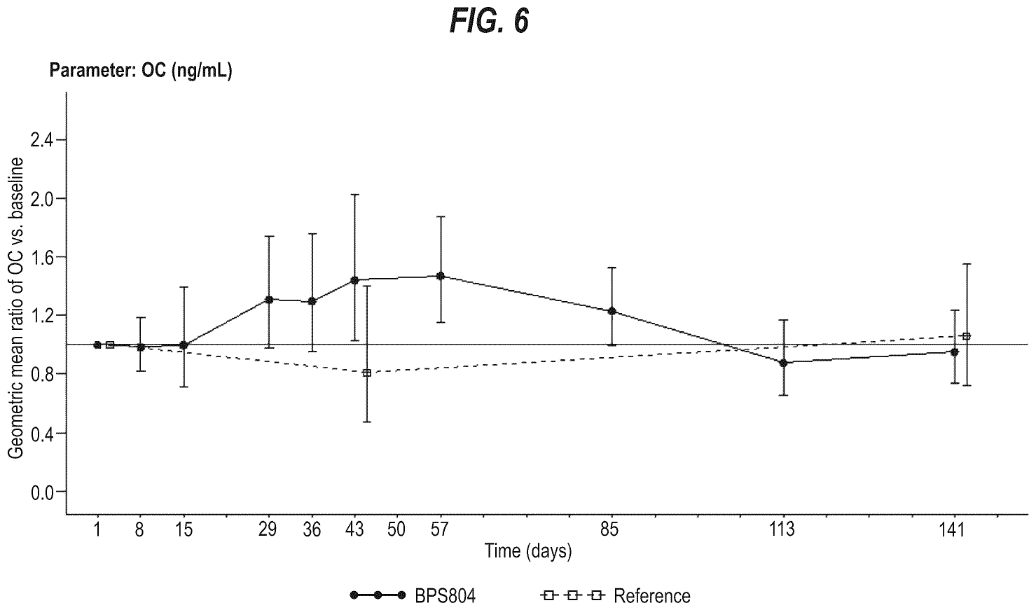

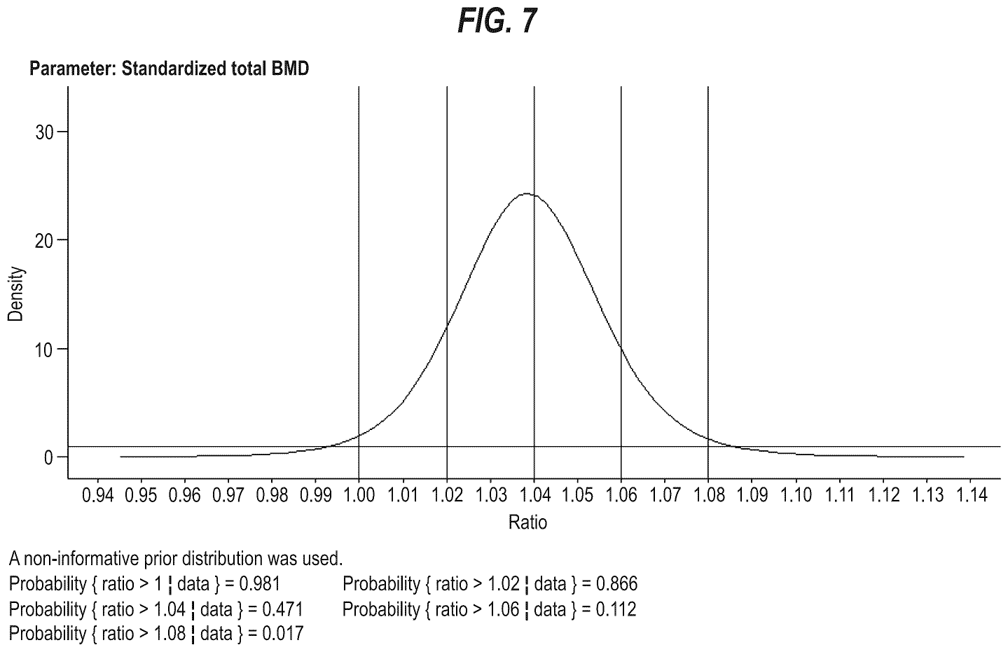

The Bayesian analysis of change from baseline for PINP, PICP, and BSAP showed a posterior probability of around 90% or higher for an increase of at least 70% (PINP) or 30% (PICP, BSAP). The Bayesian analysis for BMD showed a posterior probably of 98% for an increase in BMD, and 87% for an increase of at least 2%. See FIG. 3, FIG. 4, FIG. 5, FIG. 6 and FIG. 7 for details on the PINP, PICP, BSAP, OC and BMD results, respectively.

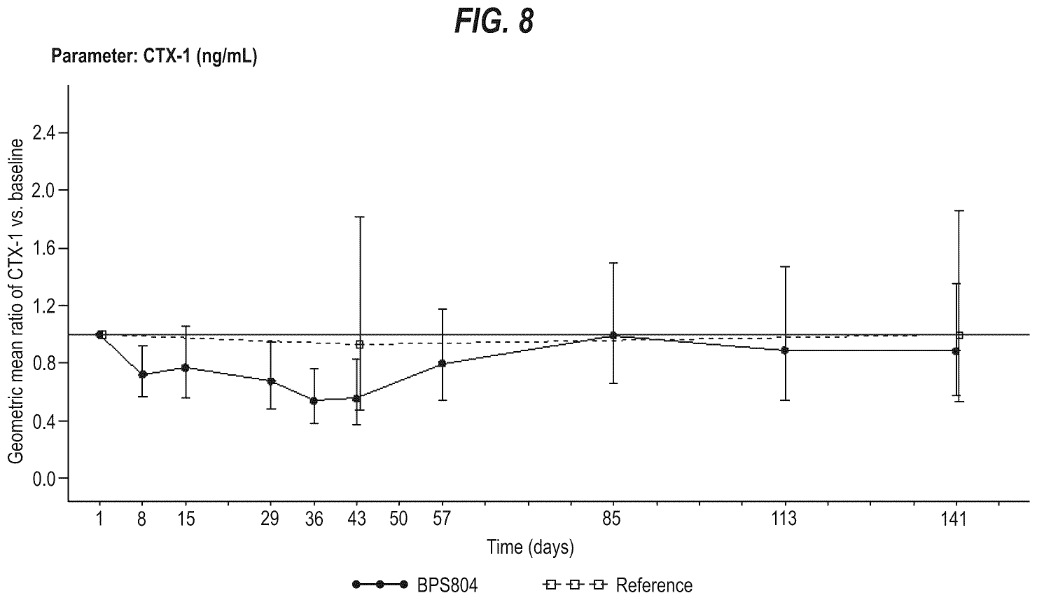

The geometric mean results on CTX-1 from Day 8 through Day43 showed a decrease in CTX-1 concentration levels which together with bone formation biomarker results supports bone anabolic effects. FIG. 8 presents the details of the values since Baseline. On Day 43, the ratio from baseline in the BPS804 group was 56% of baseline, thus a reduction by 44%. The concentration levels steadily increased from Day 50 through Day 85 with a marginal decrease on Days 113 and 141.

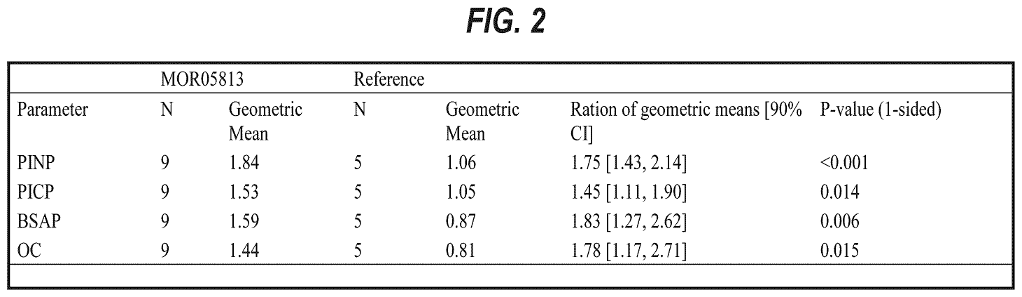

The comparison of ratios with the matching ratios in the reference group were performed as per the planned two sample t-tests (1-sided) and a P-value of below 0.1 in this comparison was considered supportive evidence for efficacy. Based on the two sample t-tests analysis, the P-values were <0.001, 0.014, 0.006, and 0.015 for PINP, PICP, BSAP, and OC, respectively, which supported the evidence for efficacy. The p-value for treatment group comparison of BMD was 0.1, also supporting the evidence that increase was larger under the anti-sclerostin antibody than in the reference group. See FIG. 2. These analyses confirmed the evidence of higher increase in the anti-sclerostin antibody group than in the reference group, for all three biomarker data and for the BMD data.

After administration of the anti-sclerostin antibody, median PINP, PICP, BSAP activity, and OC levels were increased at Day 43 by 84%, 53%, 59%, and 44%, respectively, while corresponding biomarkers remained unchanged or declined moderately in the untreated reference group. Further, after administration of the anti-sclerostin antibody, CTX-1 level was decreased by 44% at Day 43 from baseline. The increase of bone formation biomarker (PINP, PICP, BSAP and OC) levels and the reduction of bone resorption biomarker (CTX-1) level were in line with the observed increase by ˜4% in lumbar spine BMD at Day141 in the anti-sclerostin antibody treatment group, thus confirming the first clinical evidence of bone anabolic effects of an anti-sclerostin antibody in patients with osteogenesis imperfecta.

In addition, the study showed that the anti-sclerostin antibody was safe and well-tolerated in the adult patients with OI. The most commonly reported AEs were headache, influenza, arthralgia, and fatigue. None of the reported AEs were considered related to the study drug. There was one SAE (Goiter) of mild intensity reported in one patient in the reference group. This AE was considered serious because of hospitalization. The SAE resolved prior to end-of-study. The AEs gave no indication of target organ toxicity. Three fractures were reported during the study (subject 5103—Day 47, subject 5109—Day 4, Subject 5113—Day 4). There were also no clinically significant abnormalities of hematological, clinical chemistry, urinalysis, ECG or vital sign data compromising the patients' safety.

According to the clinical study results, with an osteoanabolic treatment like BPS804, bone formation or bone anabolic activity can be stimulated in patients with OI. The increase in BMD results in improved bone quality thereby leading to a reduction in the fracture rate and risk. Genotyping might be warranted to identify or predict OI patients who might benefit most from such a treatment strategy.

### Example 2

A pharmacokinetic (PK) model was developed for BPS804 along with a pharmacokinetic (PK) and pharmacodynamics (PD) (PK-PD) model for circulating sclerostin effects after BPS804 administration based on a combination of clinical trial data and publically available data on other anti-sclerostin antibodies. The PK-PD model was linked to an existing systems pharmacology model to evaluate proposed dosing regimens for BPS804. Model simulations were used to provide guidance for dose selection and dosing interval over 1-2 years of treatment for typical OI patients. These included scenarios with different dosing considerations for the first year (e.g., comparison of dosage amount and QM (i.e. monthly) vs. Q3M (i.e. quarterly) dosing) as well as considerations for subsequent years (e.g., switching from QM to Q3M dosing).

### Results:

The data demonstrated that BPS804 dosing regimens nearing maximal (>75%) inhibition of sclerostin provide near maximal responses in circulating sclerostin, bone turnover markers (BTM), and lumbar spine bone mineral density (BMD). To demonstrate this, a range of doses from 0.1 mg/kg to 20 mg/kg were simulated using QM (i.e. monthly) and Q3M (i.e. quarterly) intervals for a two-year time-course. Results were evaluated assuming full BPS804 exposure effects on sclerostin and relative to maximal inhibition. Overall, the 20 mg/kg QM dose approached the maximal sclerostin response. The maximum level of inhibition was reached by both monthly and quarterly dosing regimens, alike, and both the QM and Q3M 20 mg/kg dosing regimens reached the maximum inhibition level.

The modeling results further indicated that a BPS804 20 mg/kg QM dose would provide similar or a slightly greater 12-month BMD increase compared with Q3M dosing due to the longer sustained inhibition of sclerostin with BPS804.

Following one-year of treatment with BPS804 20 mg/kg QM, nearly the same peak maximum sclerostin response can also achieved with a 20 mg/kg dose administered quarterly. This potential change in dosing regimen after one-year of dosing could reflect the attainment of an apparent new steady-state in the bone remodeling system after one year of sclerostin inhibition therapy. Therefore, less frequent dosing after the first year of dosing may allow for maintenance of BMD increases from the first year of dosing.

In summary, the modelling data show that BPS804 20 mg/kg QM dosing is expected to provide near maximal inhibition of sclerostin that would translate into maximal BMD response. In addition, extending dosing intervals for longer-term dosing (e.g., switching to dosing every two months (Q2M) or Q3M following one-year of QM dosing) may be advantageous given the observed changes in bone turnover markers following the first year of treatment.

The patents and publications listed herein describe the general skill in the art. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting.

The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

1-25. (canceled)

26. A method for reducing risk of bone fracture associated with osteogenesis imperfecta (OI) in a human patient comprising administering to the human patient in need thereof a therapeutically effective amount of an anti-sclerostin antibody, wherein the anti-sclerostin antibody comprises:
(a) a heavy chain comprising a heavy chain variable domain (VH), wherein the VH comprises the amino acid sequence set forth in SEQ ID NO:70, and
(b) a light chain comprising a light chain variable domain (VL), wherein the VL comprises the amino acid sequence set forth in SEQ ID NO:81;
wherein the anti-sclerostin antibody is administered by injection at a dose of 20 mg per kg body weight of the human patient, and wherein the antibody is administered every 4 weeks.

27. The method of claim 26, wherein the heavy chain comprises the amino acid sequence set forth as SEQ ID NO: 172, and the light chain comprises the amino acid sequence set forth as SEQ ID NO: 173.

28. The method of claim 26, wherein reducing the risk of bone fracture associated with OI comprises reducing the fracture rate in the human patient by at least 10 percent.

29. The method of claim 28, wherein reducing the risk of bone fracture associated with OI comprises reducing the fracture rate in the human patient by at least 30 percent.

30. The method of claim 26, wherein reducing the risk of bone fracture associated with OI comprises reducing the fracture rate in the human patient compared to a control patient population.

31. The method of claim 26, wherein the bone fracture associated with OI comprises peripheral bone fractures.

32. The method of claim 26, wherein the bone fracture associated with OI comprises vertebral bone fractures.

33. The method of claim 26, wherein the bone fracture associated with OI comprises major, minor, and vertebral clinical bone fractures.

34. The method of claim 26 wherein the anti-sclerostin antibody is administered intravenously.

35. The method of claim 26, wherein the patient is a pediatric patient.

36. The method of claim 26, wherein the patient is an adult patient.

37. The method of claim 26, wherein the patient has type I OI.

38. The method of claim 26, wherein the patient has type III OI.

39. The method of claim 26, wherein the patient has type IV OI.

40. The method of claim 26, wherein the patient has one or more mutations in the COL1A1 gene.

41. The method of claim 26, wherein the patient has one or more mutations in the COL1A2 gene.

42. A pharmaceutical composition comprising an anti-sclerostin antibody in dosage unit form for reducing risk of bone fracture associated with OI in a human patient, wherein the anti-sclerostin antibody comprises:
(a) a heavy chain comprising a heavy chain variable domain (VH), wherein the VH comprises the amino acid sequence set forth in SEQ ID NO:70, and
(b) a light chain comprising a light chain variable domain (VL), wherein the VL comprises the amino acid sequence set forth in SEQ ID NO:81; and
wherein the dosage unit form comprises 10 mg-200 mg of the anti-sclerostin antibody.

43. The pharmaceutical composition of claim 42, further comprising one or more of sucrose, arginine hydrochloride, L-histidine, polysorbate 80, and hydrochloric acid.

44. The pharmaceutical composition of claim 42, wherein the composition is in a lyophilized state.


SEQUENCE LISTING (177 sequences)

SEQ ID NO: 1 - protein, 10 aa, Homo sapiens
GFTFSSYVMN

SEQ ID NO: 2 - protein, 10 aa, Homo sapiens
GFTFRSHWLS

SEQ ID NO: 3 - protein, 10 aa, Homo sapiens
GFTFSSYVMN

SEQ ID NO: 4 - protein, 10 aa, Homo sapiens
GFTFRSHWLS

SEQ ID NO: 5 - protein, 10 aa, Homo sapiens
GFTFRSHWLS

SEQ ID NO: 6 - protein, 10 aa, Homo sapiens
GFTFRSHWLS

SEQ ID NO: 7 - protein, 10 aa, Homo sapiens
GFTFRSHWLS

SEQ ID NO: 8 - protein, 10 aa, Homo sapiens
GFTFRSHWLS

SEQ ID NO: 9 - protein, 10 aa, Homo sapiens
GFTFRSHWLS

SEQ ID NO: 10 - protein, 10 aa, Homo sapiens
GFTFRSHWLS

SEQ ID NO: 11 - protein, 10 aa, Homo sapiens
GFTFRSHWLS

SEQ ID NO: 12 - protein, 20 aa, Homo sapiens
WVSFISGDSSNTYYADSVKG

SEQ ID NO: 13 - protein, 20 aa, Homo sapiens
WVSNINYDGSSTYYADSVKG

SEQ ID NO: 14 - protein, 20 aa, Homo sapiens
WVSFISGDSSNTYYADSVKG

SEQ ID NO: 15 - protein, 20 aa, Homo sapiens
WVSNINYDGSSTYYADSVKG

SEQ ID NO: 16 - protein, 19 aa, Homo sapiens
WVSVTGVHGDTYYADSVKG

SEQ ID NO: 17 - protein, 19 aa, Homo sapiens
WVSVIGNWGDTYYADSVKG

SEQ ID NO: 18 - protein, 19 aa, Homo sapiens
WVSVTTHQGYTYYADSVKG

SEQ ID NO: 19 - protein, 19 aa, Homo sapiens
WVSATNRYGYTYYADSVKG

SEQ ID NO: 20 - protein, 20 aa, Homo sapiens
WVSNINYDGSSTYYADSVKG

SEQ ID NO: 21 - protein, 19 aa, Homo sapiens
WVSVITPYGDTYYADSVKG

SEQ ID NO: 22 - protein, 19 aa, Homo sapiens
WVSVITPYGDTYYADSVKG

SEQ ID NO: 23 - protein, 15 aa, Homo sapiens
TFMHGHLGGGLSMDF

SEQ ID NO: 24 - protein, 8 aa, Homo sapiens
DTYLHFDY

SEQ ID NO: 25 - protein, 15 aa, Homo sapiens
TFMHGHLGGGLSMDF

SEQ ID NO: 26 - protein, 8 aa, Homo sapiens
DTYLHFDY

SEQ ID NO: 27 - protein, 8 aa, Homo sapiens
DTYLHFDY

SEQ ID NO: 28 - protein, 8 aa, Homo sapiens
DTYLHFDY

SEQ ID NO: 29 - protein, 8 aa, Homo sapiens
DTYLHFDY

SEQ ID NO: 30 - protein, 8 aa, Homo sapiens
DTYLHFDY

SEQ ID NO: 31 - protein, 8 aa, Homo sapiens
DTYLHFDY

SEQ ID NO: 32 - protein, 8 aa, Homo sapiens
DTYLHFDY

SEQ ID NO: 33 - protein, 8 aa, Homo sapiens
DTYLHFDY

SEQ ID NO: 34 - protein, 11 aa, Homo sapiens
SGDNIGSFYVH

SEQ ID NO: 35 - protein, 20 aa, Homo sapiens
WVSNINYDGSSTYYADSVKG

SEQ ID NO: 36 - protein, 11 aa, Homo sapiens
SGDNIGSFYVH

SEQ ID NO: 37 - protein, 14 aa, Homo sapiens
TGTSSDVGDINDVS

SEQ ID NO: 38 - protein, 14 aa, Homo sapiens
TGTSSDVGDINDVS

SEQ ID NO: 39 - protein, 14 aa, Homo sapiens
TGTSSDVGDINDVS

SEQ ID NO: 40 - protein, 14 aa, Homo sapiens
TGTSSDVGDINDVS

SEQ ID NO: 41 - protein, 14 aa, Homo sapiens
TGTSSDVGDINDVS

SEQ ID NO: 42 - protein, 14 aa, Homo sapiens
TGTSSDVGDINDVS

SEQ ID NO: 43 - protein, 14 aa, Homo sapiens
TGTSSDVGDINDVS

SEQ ID NO: 44 - protein, 14 aa, Homo sapiens
TGTSSDVGDINDVS

SEQ ID NO: 45 - protein, 11 aa, Homo sapiens
LVIYDDNNRPS

SEQ ID NO: 46 - protein, 11 aa, Homo sapiens
LMIYDVNNRPS

SEQ ID NO: 47 - protein, 11 aa, Homo sapiens
LVIYDDNNRPS

SEQ ID NO: 48 - protein, 11 aa, Homo sapiens
LMIYDVNNRPS

SEQ ID NO: 49 - protein, 11 aa, Homo sapiens
LMIYDVNNRPS

SEQ ID NO: 50 - protein, 11 aa, Homo sapiens
LMIYDVNNRPS

SEQ ID NO: 51 - protein, 11 aa, Homo sapiens
LMIYDVNNRPS

SEQ ID NO: 52 - protein, 11 aa, Homo sapiens
LMIYDVNNRPS

SEQ ID NO: 53 - protein, 11 aa, Homo sapiens
LMIYDVNNRPS

SEQ ID NO: 54 - protein, 11 aa, Homo sapiens
LMIYDVNNRPS

SEQ ID NO: 55 - protein, 11 aa, Homo sapiens
LMIYDVNNRPS

SEQ ID NO: 56 - protein, 10 aa, Homo sapiens
GSWAGSSGSY

SEQ ID NO: 57 - protein, 10 aa, Homo sapiens
SSYGESLTSY

SEQ ID NO: 58 - protein, 10 aa, Homo sapiens
ASWTGVEPDY

SEQ ID NO: 59 - protein, 10 aa, Homo sapiens
QSYAGSYLSE

SEQ ID NO: 60 - protein, 10 aa, Homo sapiens
SSYGESLTSY

SEQ ID NO: 61 - protein, 10 aa, Homo sapiens
SSYGESLTSY

SEQ ID NO: 62 - protein, 10 aa, Homo sapiens
SSYGESLTSY

SEQ ID NO: 63 - protein, 10 aa, Homo sapiens
SSYGESLTSY

SEQ ID NO: 64 - protein, 10 aa, Homo sapiens
STYDGPGLSE

SEQ ID NO: 65 - protein, 10 aa, Homo sapiens
SSYGESLTSY

SEQ ID NO: 66 - protein, 10 aa, Homo sapiens
SSYGESLTSY

SEQ ID NO: 67 - protein, 124 aa, Homo sapiens
QVQLVESGGGLVQPGGSLRLSCAASGFTFSSYVMNWVRQAPGKGLEWVSFISGDSSNTYYADSVKGRFTISRDNSKNTLYLQMNSLRAEDTAVYYCARTFMHGHLGGGLSMDFWGQGTLVTVSS

SEQ ID NO: 68 - protein, 117 aa, Homo sapiens
QVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSS

SEQ ID NO: 69 - protein, 124 aa, Homo sapiens; identical to SEQ ID NO: 67

SEQ ID NO: 70 - protein, 117 aa, Homo sapiens; identical to SEQ ID NO: 68

SEQ ID NO: 71 - protein, 116 aa, Homo sapiens
QVQLVESGGGLVQPGGSLRLSCAASGFTFRSHWLSWVRQAPGKGLEWVSVTGVHGDTYYADSVKGRFTISRDNSKNTLYLQMNSLRAEDTAVYYCARDTYLHFDYWGQGTLVTVSS

SEQ ID NO: 72 - protein, 116 aa, Homo sapiens
QVQLVESGGGLVQPGGSLRLSCAASGFTFRSHWLSWVRQAPGKGLEWVSVIGNWGDTYYADSVKGRFTISRDNSKNTLYLQMNSLRAEDTAVYYCARDTYLHFDYWGQGTLVTVSS

SEQ ID NO: 73 - protein, 116 aa, Homo sapiens
QVQLVESGGGLVQPGGSLRLSCAASGFTFRSHWLSWVRQAPGKGLEWVSVTTHQGYTYYADSVKGRFTISRDNSKNTLYLQMNSLRAEDTAVYYCARDTYLHFDYWGQGTLVTVSS

SEQ ID NO: 74 - protein, 116 aa, Homo sapiens
QVQLVESGGGLVQPGGSLRLSCAASGFTFRSHWLSWVRQAPGKGLEWVSATNRYGYTYYADSVKGRFTISRDNSKNTLYLQMNSLRAEDTAVYYCARDTYLHFDYWGQGTLVTVSS

SEQ ID NO: 75 - protein, 117 aa, Homo sapiens; identical to SEQ ID NO: 68

SEQ ID NO: 76 - protein, 116 aa, Homo sapiens
QVQLVESGGGLVQPGGSLRLSCAASGFTFRSHWLSWVRQAPGKGLEWVSVITPYGDTYYADSVKGRFTISRDNSKNTLYLQMNSLRAEDTAVYYCARDTYLHFDYWGQGTLVTVSS

SEQ ID NO: 77 - protein, 116 aa, Homo sapiens; identical to SEQ ID NO: 76

SEQ ID NO: 78 - protein, 110 aa, Homo sapiens
DIELTQPPSVSVAPGQTARISCSGDNIGSFYVHWYQQKPGQAPVLVIYDDNNRPSGIPERFSGSNSGNTATLTISGTQAEDEADYYCGSWAGSSGSYVFGGRTKLTVLGQ

SEQ ID NO: 79 - protein, 113 aa, Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCSSYGESLTSYVFGGGTKLTVLGQ

SEQ ID NO: 80 - protein, 110 aa, Homo sapiens
DIELTQPPSVSVAPGQTARISCSGDNIGSFYVHWYQQKPGQAPVLVIYDDNNRPSGIPERFSGSNSGNTATLTISGTQAEDEADYYCASWTGVEPDYVFGGGTKLTVLGQ

SEQ ID NO: 81 - protein, 113 aa, Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQ

SEQ ID NO: 82 - protein, 113 aa, Homo sapiens; identical to SEQ ID NO: 79

SEQ ID NO: 83 - protein, 113 aa, Homo sapiens; identical to SEQ ID NO: 79

SEQ ID NO: 84 - protein, 113 aa, Homo sapiens; identical to SEQ ID NO: 79

SEQ ID NO: 85 - protein, 113 aa, Homo sapiens; identical to SEQ ID NO: 79

SEQ ID NO: 86 - protein, 113 aa, Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCSTYDGPGLSEVFGGGTKLTVLGQ

SEQ ID NO: 87 - protein, 113 aa, Homo sapiens; identical to SEQ ID NO: 79

SEQ ID NO: 88 - protein, 113 aa, Homo sapiens; identical to SEQ ID NO: 86

SEQ ID NO: 89 - DNA, 372 bp, Homo sapiens
  source 1..372: mol_type = other DNA
caggtgcaattggtggaaagcggcggcggcctggtgcaaccgggcggcagcctgcgtctgagctgcgcggcctccggatttaccttttcttcttatgttatgaattgggtgcgccaagcccctgggaagggtctcgagtgggtgagctttatctctggtgattctagcaatacctattatgcggatagcgtgaaaggccgttttaccatttcacgtgataattcgaaaaacaccctgtatctgcaaatgaacagcctgcgtgcggaagatacggccgtgtattattgcgcgcgtacttttatgcatggtcatcttggtggtggtctttctatggatttttggggccaaggcaccctggtgacggttagctca

SEQ ID NO: 90 - DNA, 351 bp, Homo sapiens
  source 1..351: mol_type = other DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcaatatcaattatgatggtagctctacctattatgcggatagcgtgaaaggccgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 91 - DNA, 372 bp, Homo sapiens; identical to SEQ ID NO: 89
  source 1..372: mol_type = other DNA

SEQ ID NO: 92 - DNA, 351 bp, Homo sapiens; identical to SEQ ID NO: 90
  source 1..351: mol_type = other DNA

SEQ ID NO: 93 - DNA, 348 bp, Homo sapiens
  source 1..348: mol_type = other DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgttactggtgttcatggtgatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 94 - DNA, 348 bp, Homo sapiens
  source 1..348: mol_type = other DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgttattggtaattggggtgatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 95 - DNA, 348 bp, Homo sapiens
  source 1..348: mol_type = other DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgttactactcatcagggttatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 96 - DNA, 348 bp, Homo sapiens
  source 1..348: mol_type = other DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgctactaatcgttatggttatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 97 - DNA, 351 bp, Homo sapiens; identical to SEQ ID NO: 90
  source 1..351: mol_type = other DNA

SEQ ID NO: 98 - DNA, 348 bp, Homo sapiens
  source 1..348: mol_type = other DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgttattactccttatggtgatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 99 - DNA, 348 bp, Homo sapiens; identical to SEQ ID NO: 98
  source 1..348: mol_type = other DNA

SEQ ID NO: 100 - DNA, 330 bp, Homo sapiens
  source 1..330: mol_type = other DNA
gatatcgaactgacccagccgccttcagtgagcgttgcaccaggtcagaccgcgcgtatctcgtgtagcggcgataatattggttctttttatgttcattggtaccagcagaaacccgggcaggcgccagttcttgtgatttatgatgataataatcgtccctcaggcatcccggaacgctttagcggatccaacagcggcaacaccgcgaccctgaccattagcggcactcaggcggaagacgaagcggattattattgcggttcttgggctggttcttctggttcttatgtgtttggcggccgcacgaagttaaccgttcttggccag

SEQ ID NO: 101 - DNA, 339 bp, Homo sapiens
  source 1..339: mol_type = other DNA
gatatcgcactgacccagccagcttcagtgagcggctcaccaggtcagagcattaccatctcgtgtacgggtactagcagcgatgttggtgatattaatgatgtgtcttggtaccagcagcatcccgggaaggcgccgaaacttatgatttatgatgttaataatcgtccctcaggcgtgagcaaccgttttagcggatccaaaagcggcaacaccgcgagcctgaccattagcggcctgcaagcggaagacgaagcggattattattgctcttcttatggtgagtctcttacttcttatgtgtttggcggcggcacgaagttaaccgttcttggccag

SEQ ID NO: 102 - DNA, 330 bp, Homo sapiens
  source 1..330: mol_type = other DNA
gatatcgaactgacccagccgccttcagtgagcgttgcaccaggtcagaccgcgcgtatctcgtgtagcggcgataatattggttctttttatgttcattggtaccagcagaaacccgggcaggcgccagttcttgtgatttatgatgataataatcgtccctcaggcatcccggaacgctttagcggatccaacagcggcaacaccgcgaccctgaccattagcggcactcaggcggaagacgaagcggattattattgcgcttcttggactggtgttgagcctgattatgtgtttggcggcggcacgaagttaaccgttcttggccag

SEQ ID NO: 103 - DNA, 339 bp, Homo sapiens
  source 1..339: mol_type = other DNA
gatatcgcactgacccagccagcttcagtgagcggctcaccaggtcagagcattaccatctcgtgtacgggtactagcagcgatgttggtgatattaatgatgtgtcttggtaccagcagcatcccgggaaggcgccgaaacttatgatttatgatgttaataatcgtccctcaggcgtgagcaaccgttttagcggatccaaaagcggcaacaccgcgagcctgaccattagcggcctgcaagcggaagacgaagcggattattattgccagtcttatgctggttcttatctttctgaggtgtttggcggcggcacgaagttaaccgttcttggccag

SEQ ID NO: 104 - DNA, 339 bp, Homo sapiens; identical to SEQ ID NO: 101
  source 1..339: mol_type = other DNA

SEQ ID NO: 105 - DNA, 339 bp, Homo sapiens; identical to SEQ ID NO: 101
  source 1..339: mol_type = other DNA

SEQ ID NO: 106 - DNA, 339 bp, Homo sapiens; identical to SEQ ID NO: 101
  source 1..339: mol_type = other DNA

SEQ ID NO: 107 - DNA, 339 bp, Homo sapiens; identical to SEQ ID NO: 101
  source 1..339: mol_type = other DNA

SEQ ID NO: 108 - DNA, 339 bp, Homo sapiens
  source 1..339: mol_type = other DNA
gatatcgcactgacccagccagcttcagtgagcggctcaccaggtcagagcattaccatctcgtgtacgggtactagcagcgatgttggtgatattaatgatgtgtcttggtaccagcagcatcccgggaaggcgccgaaacttatgatttatgatgttaataatcgtccctcaggcgtgagcaaccgttttagcggatccaaaagcggcaacaccgcgagcctgaccattagcggcctgcaagcggaagacgaagcggattattattgctctacttatgatggtcctggtctttctgaggtgtttggcggcggcacgaagttaaccgttcttggccag

SEQ ID NO: 109 - DNA, 339 bp, Homo sapiens; identical to SEQ ID NO: 101
  source 1..339: mol_type = other DNA

SEQ ID NO: 110 - DNA, 339 bp, Homo sapiens; identical to SEQ ID NO: 108
  source 1..339: mol_type = other DNA

SEQ ID NO: 111 - protein, 469 aa, Homo sapiens
MAWVWTLPFLMAAAQSVQAQVQLVESGGGLVQPGGSLRLSCAASGFTFSSYVMNWVRQAPGKGLEWVSFISGDSSNTYYADSVKGRFTISRDNSKNTLYLQMNSLRAEDTAVYYCARTFMHGHLGGGLSMDFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 112 - protein, 462 aa, Homo sapiens
MAWVWTLPFLMAAAQSVQAQVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 113 - protein, 469 aa, Homo sapiens; identical to SEQ ID NO: 111

SEQ ID NO: 114 - protein, 462 aa, Homo sapiens; identical to SEQ ID NO: 112

SEQ ID NO: 115 - protein, 461 aa, Homo sapiens
MAWVWTLPFLMAAAQSVQAQVQLVESGGGLVQPGGSLRLSCAASGFTFRSHWLSWVRQAPGKGLEWVSVTGVHGDTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 116 - protein, 461 aa, Homo sapiens
MAWVWTLPFLMAAAQSVQAQVQLVESGGGLVQPGGSLRLSCAASGFTFRSHWLSWVRQAPGKGLEWVSVIGNWGDTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 117 - protein, 461 aa, Homo sapiens
MAWVWTLPFLMAAAQSVQAQVQLVESGGGLVQPGGSLRLSCAASGFTFRSHWLSWVRQAPGKGLEWVSVTTHQGYTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 118 - protein, 465 aa, Homo sapiens
MKHLWFFLLLVAAPRWVLSQVQLVESGGGLVQPGGSLRLSCAASGFTFRSHWLSWVRQAPGKGLEWVSATNRYGYTYYADSVKGRFTISRDNSKNTLYLQMNSLRAEDTAVYYCARDTYLHFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 119 - protein, 462 aa, Homo sapiens; identical to SEQ ID NO: 112

SEQ ID NO: 120 - protein, 461 aa, Homo sapiens
MAWVWTLPFLMAAAQSVQAQVQLVESGGGLVQPGGSLRLSCAASGFTFRSHWLSWVRQAPGKGLEWVSVITPYGDTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 121 - protein, 461 aa, Homo sapiens; identical to SEQ ID NO: 120

SEQ ID NO: 122 - protein, 130 aa, Homo sapiens
MSVLTQVLALLLLWLTGTRCDIELTQPPSVSVAPGQTARISCSGDNIGSFYVHWYQQKPGQAPVLVIYDDNNRPSGIPERFSGSNSGNTATLTISGTQAEDEADYYCGSWAGSSGSYVFGGRTKLTVLGQ

SEQ ID NO: 123 - protein, 237 aa, Homo sapiens
MSVLTQVLALLLLWLTGTRCDIALTQPASVSGSPGQSITISCTGTSSDVGDINDVSWYQQHPGKAPKLMIYDVNNRPSGVSNRFSGSKSGNTASLTISGLQAEDEADYYCSSYGESLTSYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 124 - protein, 234 aa, Homo sapiens
MSVLTQVLALLLLWLTGTRCDIELTQPPSVSVAPGQTARISCSGDNIGSFYVHWYQQKPGQAPVLVIYDDNNRPSGIPERFSGSNSGNTATLTISGTQAEDEADYYCASWTGVEPDYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 125 - protein, 237 aa, Homo sapiens
MSVLTQVLALLLLWLTGTRCDIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 126 - protein, 237 aa, Homo sapiens; identical to SEQ ID NO: 123

SEQ ID NO: 127 - protein, 237 aa, Homo sapiens; identical to SEQ ID NO: 123

SEQ ID NO: 128 - protein, 237 aa, Homo sapiens; identical to SEQ ID NO: 123

SEQ ID NO: 129 - protein, 237 aa, Homo sapiens; identical to SEQ ID NO: 123

SEQ ID NO: 130 - protein, 237 aa, Homo sapiens
MSVLTQVLALLLLWLTGTRCDIALTQPASVSGSPGQSITISCTGTSSDVGDINDVSWYQQHPGKAPKLMIYDVNNRPSGVSNRFSGSKSGNTASLTISGLQAEDEADYYCSTYDGPGLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 131 - protein, 237 aa, Homo sapiens; identical to SEQ ID NO: 123

SEQ ID NO: 132 - protein, 237 aa, Homo sapiens; identical to SEQ ID NO: 130

SEQ ID NO: 133 - DNA, 1410 bp, Homo sapiens
  source 1..1410: mol_type = other DNA
atggcttgggtgtggaccttgccattcctgatggcagctgcccaaagcgtgcaggcccaggtgcagctggtcgagtctggcggcggactggtgcagcctggcggcagcctgagactgagctgcgccgccagcggcttcaccttcagcagctacgtgatgaactgggtgcggcaggcccctggcaagggcctggagtgggtgtccttcatcagcggcgacagcagcaacacctactacgccgacagcgtgaagggccggttcaccatcagccgggacaacagcaagaacaccctgtacctgcagatgaacagcctgcgggccgaggacaccgccgtgtactactgcgcccggaccttcatgcacggccacctgggcggaggactgagcatggatttctggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 134 - DNA, 1389 bp, Homo sapiens
  source 1..1389: mol_type = other DNA
atggcttgggtgtggaccttgccattcctgatggcagctgcccaaggtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccaacatcaactacgacggcagcagcacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 135 - DNA, 1410 bp, Homo sapiens; identical to SEQ ID NO: 133
  source 1..1410: mol_type = other DNA

SEQ ID NO: 136 - DNA, 1389 bp, Homo sapiens; identical to SEQ ID NO: 134
  source 1..1389: mol_type = other DNA

SEQ ID NO: 137 - DNA, 1386 bp, Homo sapiens
  source 1..1386: mol_type = other DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgtgaccggcgtgcacggcgacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 138 - DNA, 1386 bp, Homo sapiens
  source 1..1386: mol_type = other DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgtgatcggcaactggggcgacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 139 - DNA, 1386 bp, Homo sapiens
  source 1..1386: mol_type = other DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgtgaccacccaccagggctacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 140 - DNA, 1386 bp, Homo sapiens
  source 1..1386: mol_type = other DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgccaccaacagatacggctacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 141 - DNA, 1389 bp, Homo sapiens; identical to SEQ ID NO: 134
  source 1..1389: mol_type = other DNA

SEQ ID NO: 142 - DNA, 1386 bp, Homo sapiens
  source 1..1386: mol_type = other DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgtgatcaccccctacggcgacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 143 - DNA, 1386 bp, Homo sapiens; identical to SEQ ID NO: 142
  source 1..1386: mol_type = other DNA

SEQ ID NO: 144 - DNA, 393 bp, Homo sapiens
  source 1..393: mol_type = other DNA
atgaaaaagacagctatcgcgattgcagtggcactggctggtttcgctaccgtagcgcaggccgatatcgaactgacccagccgccttcagtgagcgttgcaccaggtcagaccgcgcgtatctcgtgtagcggcgataatattggttctttttatgttcattggtaccagcagaaacccgggcaggcgccagttcttgtgatttatgatgataataatcgtccctcaggcatcccggaacgctttagcggatccaacagcggcaacaccgcgaccctgaccattagcggcactcaggcggaagacgaagcggattattattgcggttcttgggctggttcttctggttcttatgtgtttggcggccgcacgaagttaaccgttcttggccag

SEQ ID NO: 145 - DNA, 714 bp, Homo sapiens
  source 1..714: mol_type = other DNA
atgagtgtgctcactcaggtcctggcgttgctgctgctgtggcttacaggtacgcgttgcgacatcgccctgacccagcccgccagcgtgagcggcagccctggccagagcatcaccatcagctgcaccggcaccagcagcgacgtgggcgacatcaacgacgtgagctggtatcagcagcaccccggcaaggcccccaagctgatgatctacgacgtgaacaaccggcccagcggcgtgagcaaccggttcagcggcagcaagagcggcaacaccgccagcctgaccatcagcggcctccaggccgaggacgaggccgactactactgcagcagctacggcgagagcctgaccagctacgtgtttggcggcggaaccaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 146 - DNA, 705 bp, Homo sapiens
  source 1..705: mol_type = other DNA
atgagtgtgctcactcaggtcctggcgttgctgctgctgtggcttacaggtacgcgttgcgacatcgagctgacccagccccccagcgtgagcgtggcccctggccagaccgcccggatcagctgcagcggcgacaacatcggcagcttctacgtgcactggtatcagcagaagcccggccaggcccccgtgctggtgatctacgacgacaacaaccggcccagcggcatccccgagcggttcagcggcagcaacagcggcaacaccgccaccctgaccatcagcggcacccaggccgaggacgaggccgactactactgcgccagctggaccggcgtggagcccgactacgtgtttggcggcggaacaaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 147 - DNA, 714 bp, Homo sapiens
  source 1..714: mol_type = other DNA
atgagtgtgctcactcaggtcctggcgttgctgctgctgtggcttacaggtacgcgttgcgacatcgccctgacccagcccgccagcgtgagcggcagccctggccagagcatcaccatcagctgcaccggcaccagcagcgacgtgggcgacatcaacgacgtgagctggtatcagcagcaccccggcaaggcccccaagctgatgatctacgacgtgaacaaccggcccagcggcgtgagcaaccggttcagcggcagcaagagcggcaacaccgccagcctgaccatcagcggcctccaggccgaggacgaggccgactactactgccagagctacgccggcagctacctgagcgaggtgttcggcggagggaccaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 148 - DNA, 714 bp, Homo sapiens; identical to SEQ ID NO: 145
  source 1..714: mol_type = other DNA

SEQ ID NO: 149 - DNA, 714 bp, Homo sapiens; identical to SEQ ID NO: 145
  source 1..714: mol_type = other DNA

SEQ ID NO: 150 - DNA, 714 bp, Homo sapiens; identical to SEQ ID NO: 145
  source 1..714: mol_type = other DNA

SEQ ID NO: 151 - DNA, 714 bp, Homo sapiens; identical to SEQ ID NO: 145
  source 1..714: mol_type = other DNA

SEQ ID NO: 152 - DNA, 714 bp, Homo sapiens
  source 1..714: mol_type = other DNA
atgagtgtgctcactcaggtcctggcgttgctgctgctgtggcttacaggtacgcgttgcgacatcgccctgacccagcccgccagcgtgagcggcagccctggccagagcatcaccatcagctgcaccggcaccagcagcgacgtgggcgacatcaacgacgtgagctggtatcagcagcaccccggcaaggcccccaagctgatgatctacgacgtgaacaaccggcccagcggcgtgagcaaccggttcagcggcagcaagagcggcaacaccgccagcctgaccatcagcggcctccaggccgaggacgaggccgactactactgcagcacctacgacggccctggcctgagcgaggtgttcggcggagggaccaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 153 - DNA, 714 bp, Homo sapiens; identical to SEQ ID NO: 152
  source 1..714: mol_type = other DNA

SEQ ID NO: 154 - DNA, 714 bp, Homo sapiens; identical to SEQ ID NO: 152
  source 1..714: mol_type = other DNA

SEQ ID NO: 155 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 156 - protein, 14 aa, Homo sapiens
ARLLNAIGRGKWWR

SEQ ID NO: 157 - protein, 15 aa, Homo sapiens
RLVASCKCKRLTRFH

SEQ ID NO: 158 - protein, 13 aa, synthetic construct
  REGION 1..13: note = OTHER INFORMATION: Synthetic
GGGSGGGGSGGGG

SEQ ID NO: 159 - protein, 19 aa, synthetic construct
  REGION 1..19: note = OTHER INFORMATION: Synthetic
GGGGSGGGGSGGGGSGGGG

SEQ ID NO: 160 - DNA, 21 bp, synthetic construct
  misc_feature 1..21: note = OTHER INFORMATION: Synthetic
  source 1..21: mol_type = other DNA
taaattatcataaagtcctaa

SEQ ID NO: 161 - DNA, 21 bp, synthetic construct
  misc_feature 1..21: note = OTHER INFORMATION: Synthetic
  source 1..21: mol_type = other DNA
aggactttatgataatttatt

SEQ ID NO: 162 - DNA, 21 bp, synthetic construct
  misc_feature 1..21: note = OTHER INFORMATION: Synthetic
  source 1..21: mol_type = other DNA
atagtggttaaataactccag

SEQ ID NO: 163 - DNA, 21 bp, synthetic construct
  misc_feature 1..21: note = OTHER INFORMATION: Synthetic
  source 1..21: mol_type = other DNA
ggagttatttaaccactattt

SEQ ID NO: 164 - DNA, 21 bp, synthetic construct
  misc_feature 1..21: note = OTHER INFORMATION: Synthetic
  source 1..21: mol_type = other DNA
taaattctcgtgatgtgccat

SEQ ID NO: 165 - DNA, 21 bp, synthetic construct
  misc_feature 1..21: note = OTHER INFORMATION: Synthetic
  source 1..21: mol_type = other DNA
ggcacatcacgagaatttatt

SEQ ID NO: 166 - DNA, 21 bp, synthetic construct
  misc_feature 1..21: note = OTHER INFORMATION: Synthetic
  source 1..21: mol_type = other DNA
tttcttatagcacagctggtt

SEQ ID NO: 167 - DNA, 21 bp, synthetic construct
  misc_feature 1..21: note = OTHER INFORMATION: Synthetic
  source 1..21: mol_type = other DNA
ccagctgtgctataagaaatt

SEQ ID NO: 168 - DNA, 21 bp, synthetic construct
  misc_feature 1..21: note = OTHER INFORMATION: Synthetic
  source 1..21: mol_type = other DNA
tagacctttccatccacgctg

SEQ ID NO: 169 - DNA, 21 bp, synthetic construct
  misc_feature 1..21: note = OTHER INFORMATION: Synthetic
  source 1..21: mol_type = other DNA
gcgtggatggaaaggtctatt

SEQ ID NO: 170 - DNA, 29 bp, synthetic construct
  misc_feature 1..29: note = OTHER INFORMATION: Synthetic
  source 1..29: mol_type = other DNA
atgcagctcccactggccctgtgtcttgt

SEQ ID NO: 171 - DNA, 30 bp, synthetic construct
  misc_feature 1..30: note = OTHER INFORMATION: Synthetic
  source 1..30: mol_type = other DNA
aatcaggccgagctggagaacgcctactag

SEQ ID NO: 172 - protein, 443 aa, Homo sapiens
QVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 173 - protein, 217 aa, synthetic construct
  REGION 1..217: note = Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 174 - protein, 26 aa, Homo sapiens
CGPARLLPNAIGRGKWWRPSGPDFRC

SEQ ID NO: 175 - protein, 60 aa, Homo sapiens
DVSEYCRELHFTRSAKPVTELVCSGQCGPARWWRPSGPDFRCIPDRYRLVASCKCKRLTR

SEQ ID NO: 176 - protein, 133 aa, synthetic construct
  REGION 1..133: note = Synthetic Polypeptide
  REGION 1..133: note = MISC_FEATURE - Humanized Antibody Sequence
MDTRAPTQLLGLLLLWLPGATFAQVLTQSPSSLSASVGDRVTITCQSSQSVYDNNWLAWYQQKPGKAPKLLIYDASDLASGVPSRFSGSGSGTDFTLTISSLQPEDFATYYCQGAYNDVIYAFGQGTKVEIKR

SEQ ID NO: 177 - protein, 131 aa, synthetic construct
  REGION 1..131: note = Synthetic Polypeptide
  REGION 1..131: note = MISC_FEATURE - Humanized Antibody Sequence
METGLRWLLLVAVLKGVHCEVQLLESGGGLVQPGGSLRLSCAASGFSLSSYWMNWVRQAPGKGLEWVGTIDSGGRTDYASWAKGRFTISRDNSKNTMYLQMNSLRAEDTARYYCARNWNLWGQGTLVTVSS